SEQUENCE LISTING (18 sequences)

SEQ ID NO: 1 - protein, 10 aa, Homo sapiens
NSIALKNNTL

SEQ ID NO: 2 - protein, 14 aa, Homo sapiens
NSNNICINDNINTL

SEQ ID NO: 3 - protein, 15 aa, Homo sapiens
GAITVGNKNNDKLTL

SEQ ID NO: 4 - protein, 14 aa, Homo sapiens
NSNNICIDDNINTL

SEQ ID NO: 5 - protein, 12 aa, Homo sapiens
GDICIKDSINTL

SEQ ID NO: 6 - protein, 221 aa, Adenovirus
RGSHHHHHHGSKNSIALKNNTLWTGPKPEANSIIEYGKQNPDSKLTLILVKNGGIVNGYVTLMGASDYVNTLFKNKNVSINVELYFDATGHILPDSSSLKTDLELKYKQTADFSARGFMPSTTAYPFDLPNAGTHNENYIFGQSYYKASDGALFPLEVTVMLNKRLPDSRTSYVMTFLWSLNAGLAPETTQATLITSPFTFSYIREDDGGGSGGGSGGGSC

SEQ ID NO: 7 - protein, 221 aa, Adenovirus
RGSHHHHHHGSKNSIALKNNTLWTGPKPEANSIIEYGKQNPDSKLTLILVKNGGIVNGYVTLMGASDYVNTLFKNKNVSINVELYFDATGHILPDSSSLKTDLELKYKQTADFSARGFMPSTTAYPFDLPNAGTHNENFIFGQSYYKASDGALFPLEVTVMLNKRLPDSRTSYVMTFLWSLNAGLAPETTQATLITSPFTFSYIREDDGGGSGGGSGGGSC

SEQ ID NO: 8 - protein, 221 aa, Adenovirus
RGSHHHHHHGSKNSIALKNNTLWTGPKPEANSIIEYGKQNPDSKLTLILVKNGGIVNGYVTLMGASDYVNTLFKNKNVSINVELYFDATGHILPDSSSLKTDLELKYKQTADFSARGFMPSTTAYPFDLPNAGTHNENYIFGQSYYKASDGALFPLEVTVMLNKRLPDSRTSYVMTFLWSLSAGLAPETTQATLITSPFTFSYIREDDGGGSGGGSGGGSC

SEQ ID NO: 9 - protein, 221 aa, Adenovirus
RGSHHHHHHGSKNSIALKNNTLWTGPKPEANSIIEYGKQNPDSKLTLILVKNGGIVNGYVTLMGASDYVNTLFKNKNVSINVELYFDATGHILPDSSSLKTDLELKYKQTADFSARGFMPSTTAYPFDLPNAGTHNENFIFGQSYYKASDGALFPLEVTVMLNKRLPDSRTSYVMTFLWSLSAGLAPETTQATLITSPFTFSYIREDDGGGSGGGSGGGSC

SEQ ID NO: 10 - protein, 213 aa, Adenovirus
RGSKNSIALKNNTLWTGPKPEANSIIEYGKQNPDSKLTLILVKNGGIVNGYVTLMGASDYVNTLFKNKNVSINVELYFDATGHILPDSSSLKTDLELKYKQTADFSARGFMPSTTAYPFDLPNAGTHNENYIFGQSYYKASDGALFPLEVTVMLNKRLPDSRTSYVMTFLWSLNAGLAPETTQATLITSPFTFSYIREDDGGGSGGGSGGGSC

SEQ ID NO: 11 - protein, 213 aa, Adenovirus
RGSKNSIALKNNTLWTGPKPEANSIIEYGKQNPDSKLTLILVKNGGIVNGYVTLMGASDYVNTLFKNKNVSINVELYFDATGHILPDSSSLKTDLELKYKQTADFSARGFMPSTTAYPFDLPNAGTHNENFIFGQSYYKASDGALFPLEVTVMLNKRLPDSRTSYVMTFLWSLNAGLAPETTQATLITSPFTFSYIREDDGGGSGGGSGGGSC

SEQ ID NO: 12 - protein, 213 aa, Adenovirus
RGSKNSIALKNNTLWTGPKPEANSIIEYGKQNPDSKLTLILVKNGGIVNGYVTLMGASDYVNTLFKNKNVSINVELYFDATGHILPDSSSLKTDLELKYKQTADFSARGFMPSTTAYPFDLPNAGTHNENYIFGQSYYKASDGALFPLEVTVMLNKRLPDSRTSYVMTFLWSLSAGLAPETTQATLITSPFTFSYIREDDGGGSGGGSGGGSC

SEQ ID NO: 13 - protein, 213 aa, Adenovirus
RGSKNSIALKNNTLWTGPKPEANSIIEYGKQNPDSKLTLILVKNGGIVNGYVTLMGASDYVNTLFKNKNVSINVELYFDATGHILPDSSSLKTDLELKYKQTADFSARGFMPSTTAYPFDLPNAGTHNENFIFGQSYYKASDGALFPLEVTVMLNKRLPDSRTSYVMTFLWSLSAGLAPETTQATLITSPFTFSYIREDDGGGSGGGSGGGSC

SEQ ID NO: 14 - protein, 269 aa, Homo sapiens
MRGSHHHHHHGSKVSALKEKVSALKEKVSALKEKVSALKEKVSALKEGSGGGSGGGSGGGSNSIALKNNTLWTGPKPEANCIIEYGKQNPDSKLTLILVKNGGIVNGYVTLMGASDYVNTLFKNKNVSINVELYFDATGHILPDSSSLKTDLELKYKQTADFSARGFMPSTTAYPFVLPNAGTHNENYIFGQCYYKASDGALFPLEVTVMLNKRLPDSRTSYVMTFLWSLNAGLAPETTQATLITSPFTFSYIREDDGGGSGGGSGGGS

SEQ ID NO: 15 - DNA, 24 bp, Homo sapiens
tcctctcatatgtatatctccttc

SEQ ID NO: 16 - DNA, 23 bp, Homo sapiens
cttaattagctgaaatcactagt

SEQ ID NO: 17 - DNA, 813 bp, Homo sapiens
atgagaggatcgcatcaccatcaccatcacggttctaaagtgagcgcgttgaaggagaaggtctctgcattaaaagaaaaggtttcagctctaaaagagaaggtaagtgcccttaaagaaaaggtgtctgcactcaaggagggctctggtggtggtagcggtggtggctccggtggtggtagcaattccattgctctgaagaacaacacgctgtggactggtccgaaaccggaagctaactctattattgagtacggtaaacagaacccggactccaaactgactctgattctggtaaaaaacggcggcatcgttaacggttatgttactctgatgggtgcttccgactatgttaacaccctgttcaagaacaaaaacgtatctatcaacgttgagctgtatttcgacgcaaccggtcacattctgccggactcctcttctctgaaaacggacctggaactgaaatacaaacagactgctgatttctctgcccgtggtttcatgccgagcaccactgcatatccgtttgatctgccaaacgccggcactcataatgaaaactacatttttggtcagtcttactataaagcgtccgacggcgcgctgttcccgctggaagttaccgttatgctgaacaaacgcctgccggattctcgcactagctacgtgatgactttcctgtggtccctgaacgcgggcctggcaccagaaactactcaggcaaccctcataacctccccatttaccttttcctatattagagaagatgacggaggcggaagcggagggggttccggaggaggcagctgttaa

SEQ ID NO: 18 - DNA, 669 bp, Homo sapiens
atgagaggatcgcatcaccatcaccatcacggatcaaagaattccattgctctgaagaacaacacgctgtggactggtccgaaaccggaagctaactctattattgagtacggtaaacagaacccggactccaaactgactctgattctggtaaaaaacggcggcatcgttaacggttatgttactctgatgggtgcttccgactatgttaacaccctgttcaagaacaaaaacgtatctatcaacgttgagctgtatttcgacgcaaccggtcacattctgccggactcctcttctctgaaaacggacctggaactgaaatacaaacagactgctgatttctctgcccgtggtttcatgccgagcaccactgcatatccgtttgatctgccaaacgccggcactcataatgaaaactacatttttggtcagtcttactataaagcgtccgacggcgcgctgttcccgctggaagttaccgttatgctgaacaaacgcctgccggattctcgcactagctacgtgatgactttcctgtggtccctgaacgcgggcctggcaccagaaactactcaggcaaccctcataacctccccatttaccttttcctatattagagaagatgacggaggcggaagcggagggggttccggaggaggcagctgttaa

(19) United States
(12) Patent Application Publication
(10) Pub. No.: US 2020/0347101 A1
(43) Pub. Date: Nov. 5, 2020

(54) CONJUGATABLE DESMOGLEIN 2 (DSG2) BINDING PROTEINS AND USES THEREFOR

(71) Applicant: PAI LIFE SCIENCES, INC., Seattle, WA (US)

(72) Inventors: Darrick CARTER, Seattle, WA (US); Sean GRAY, Seattle, WA (US)

(21) Appl. No.: 16/964,733

(22) PCT Filed: Jan. 25, 2019
(86) PCT No.: PCT/US2019/015280

§ 371 Date: Jul. 24, 2020

Related U.S. Application Data
(60) Provisional application No. 62/621,894, filed on Jan. 25, 2018.

(51) Int. Cl. C07K 14/005; C12N 7/00; A61K 47/62; A61P 35/00; A61K 51/08; A61K 47/68; A61K 47/60; A61K 47/54; A61K 47/69; A61K 47/64

(52) U.S. Cl. CPC C07K 14/005; C12N 7/00; A61K 47/62; A61P 35/00; A61K 51/088; A61K 47/6811; A61K 38/00; A61K 47/545; A61K 47/549; A61K 47/6911; A61K 47/64; C12N 2710/10322; A61K 47/60

(57) ABSTRACT

The disclosure provides polypeptide compositions that open a tumor tight junction, comprising an adenovirus fiber polypeptide shaft domain motif; a sequence that opens a tumor tight junction; a multimerization domain; and a conjugatable moiety. In another aspect, the multimerization domain comprises a conjugatable moiety.

### CROSS-REFERENCE TO RELATED APPLICATION

This patent application claims the benefit under 35 U.S.C. § 119(e) of U.S. Provisional Patent Application No. 62/621,894 filed Jan. 25, 2018, which application is incorporated herein by reference in its entirety for all purposes.

### STATEMENT OF GOVERNMENT RIGHTS

This invention was made with U.S. Government support under grant SBIR R43 CA206607 awarded by the National Institutes of Health. The U.S. Government has certain rights in this invention.

### TECHNICAL FIELD

This patent application relates generally to proteins, such as modified desmoglein 2-binding proteins that open tight junctions, and uses and preparation thereof.

### SEQUENCE LISTING

The sequence listing associated with this application is provided in text format in lieu of a paper copy, and is hereby incorporated by reference into the specification. The name of the text file containing the sequence listing is 1001401 PC_ST25; the text file is 20.8 KB, was created on 25 Jan. 2019, and is being submitted electronically via EFS-web.

### SUMMARY

In one aspect, the present disclosure provides a polypeptide, such as a polypeptide that opens a tumor tight junction, the polypeptide comprising: an adenovirus fiber polypeptide shaft domain motif, a polypeptide sequence that induces the opening of a tumor tight junction, a multimerization domain, and a moiety for targeted conjugation, e.g., a conjugatable moiety. In another aspect, the multimerization domain comprises a conjugatable moiety.

In certain embodiments, each shaft domain motif present in the polypeptide is selected from the group consisting of an Ad3 fiber polypeptide shaft domain motif, an Ad7 fiber polypeptide shaft domain motif, an Ad11 fiber polypeptide shaft domain motif, an Ad 14 fiber polypeptide shaft domain motif, an Ad14a fiber polypeptide shaft domain motif, or any combinations thereof. Each shaft domain motif may comprise an amino acid sequence selected from the group consisting of SEQ ID NOs: 1-5.

In other embodiments, the sequence that induces opening of a tumor tight junction is a binding sequence for desmoglein 2. The binding sequence for desmoglein 2 may be derived from an adenovirus knob sequence, and in certain embodiments is derived from Ad3, Ad7, Ad11, Ad14, or Ad14a.

In other embodiments, all cysteinyl residues in the polypeptide, other than the conjugatable moiety in the case where the conjugatable moiety is a cysteinyl residue, are changed to serinyl residues. In certain embodiments, the cysteinyl residues are changed to serinyl residues. In some embodiments, the multimerization domain contains a glycine-serine linker sequence. In other embodiments, the conjugatable moiety is an amino acid residue capable of entering into a covalent conjugation reaction, e.g., a cysteinyl residue which has a conjugatable thiol group. In yet other embodiments, the multimerization domain conjugates polypeptides to form multimers, e.g., the multimerization domain conjugates two polypeptides to form a homodimer.

Optionally, the polypeptide of the present disclosure contains only a single cysteinyl residue. In one embodiment, that single cysteinyl residue is a terminal cysteinyl residue such as shown in FIG. 1Ab. Optionally, that single cysteinyl residue is joined to the remainder of the polypeptide via a linker group or linker sequence as also illustrated in FIG. 1Ab. Optionally, that single cysteinyl residue is a terminal cysteinyl residue that is joined to the remainder of the polypeptide via a linker group or linker sequence such as shown in FIG. 1Ab. The disclosure also provides for multimers of such polypeptides. The disclosure also provides for the corresponding structure where the single cysteinyl residue has entered into a reaction to form a covalent bond to a compound, and thus functioned as a conjugatable moiety which has joined together the compound and the polypeptide.

In embodiments, the polypeptide comprises an amino acid residue sequence as set forth in any one of SEQ ID Nos: 6-13.

In certain embodiments, the polypeptide further comprises one or more compounds that is or are conjugated to the polypeptide through the conjugatable moiety or the moiety for targeted conjugation, which is referred to herein as a conjugated polypeptide. In embodiments, the one or more compounds of the conjugated polypeptide are selected from the group consisting of therapeutics, diagnostics and imaging agents. In other embodiments, the one or more compounds comprise at least one therapeutic, wherein the therapeutic is selected from the group consisting of antibodies, immunoconjugates, immune stimulators, CAR T-cells, nanoparticles, chemotherapeutics, radioactive particles, viruses, vaccines, cellular immunotherapy therapeutics, gene therapy constructs, nucleic acid therapeutics and combinations thereof.

The present disclosure also provide an isolated nucleic acid encoding any of the disclosed polypeptides. Furthermore, the present disclosure provides a recombinant expression vector comprising the isolated nucleic acid and a host cell comprising the recombinant expression vector.

In another embodiment, the present disclosure provides a pharmaceutical composition, comprising a polypeptide according to any of the embodiments disclosed herein and a pharmaceutically acceptable carrier.

Also provided are methods for enhancing therapeutic treatment, or diagnosis of a disorder associated with epithelial tissue, and/or imaging epithelial tissues, comprising administering to a subject in need thereof, a therapeutic for treatment of the disorder, a diagnostic, or an imaging agent; and the pharmaceutical composition disclosed herein in an amount sufficient to enhance efficacy of the therapeutic, diagnostic, and imaging agent. In embodiments, the disorder associated with human tissue is selected from the group consisting of solid tumors, irritable bowel syndrome, inflammatory bowel disorder, Crohn's disease, ulcerative colitis, constipation, gatroesophageal reflux disease, Barrett's esophagus, chronic obstructive pulmonary disease, asthma, bronchitis, pulmonary emphysema, cystic fibrosis, interstitial lung disease, pneumonia, primary pulmonary hypertension, pulmonary embolism, pulmonary sarcoidosis, tuberculosis, pancreatitis, pancreatic duct disorders, bile duct obstruction, cholecystitis, choledocholithiasis, brain disorders, psoriasis, dermatitis, glomerulonephritis, hepatitis, diabetes, thyroid disorders, cellulitis, infection, pyelonephritis, multiple sclerosis, transplant rejection and gallstones. The epithelial disorder may be a solid tumor selected from the group consisting of breast tumors, lung tumors, colon tumors, rectal tumors, stomach tumors, prostate tumors, ovarian tumors, uterine tumors, skin tumors, endocrine tumors, cervical tumors, kidney tumors, melanomas, pancreatic tumors, liver tumors, brain tumors, head and neck tumors, nasopharyngeal tumors, gastric tumors, squamous cell carcinomas, adenocarcinomas, bladder tumors and esophageal tumors.

In embodiments, the one or more compounds comprises at least one therapeutic, wherein the therapeutic is selected from the group consisting of antibodies, immunoconjugates, viruses, nanoparticles, immune stimulators, chemotherapeutics, radioactive particle, vaccines, cellular immunotherapy therapeutics, gene therapy constructs, nucleic acid therapeutics and combinations thereof. In certain embodiments, the therapeutic comprises a chemotherapeutic or a monoclonal antibody, such as an anti-tumor monoclonal antibody. In other embodiments, the disorder associated with epithelial tissue comprises a Her-2 positive tumor.

The present disclosure also provide a method for improving delivery of a compound to an epithelial tissue, comprising contacting the epithelial tissue with one or more compounds to be delivered to the epithelial tissue; and a conjugated polypeptide of any one of disclosed embodiments or functional equivalent thereof, or a disclosed pharmaceutical composition, in an amount sufficient to direct delivery of the one or more compounds to the epithelial tissue. In certain embodiments, the one or more compounds comprises a diagnostic or an imaging agent.

The present disclosure also provide a method for improving delivery of a substance to a tissue expressing desmoglein 2 (DSG2), comprising contacting the tissue expressing DSG2 with one or more compound to be delivered to the tissue; and linked to an amount of the recombinant protein as disclosed herein, including any one of the following exemplary embodiments: Embodiment 1 is a polypeptide that opens a tumor tight junction, comprising: an adenovirus fiber polypeptide shaft domain motif, a sequence that induces the opening (either by direct binding or by indirect action) of a tumor tight junction, a multimerization domain, and a moiety for targeted conjugation, e.g., a conjugatable moiety. In another aspect, the multimerization domain comprises a conjugatable moiety. In certain embodiments, each shaft domain motif present in the polypeptide is selected from the group consisting of an Ad3 fiber polypeptide shaft domain motif, an Ad7 fiber polypeptide shaft domain motif, an Ad11 fiber polypeptide shaft domain motif, an Ad 14 fiber polypeptide shaft domain motif, an Ad14a fiber polypeptide shaft domain motif, which includes combinations thereof. In another embodiment, each shaft domain motif comprises an amino acid sequence selected from the group consisting of SEQ ID NOS: 1-5. In yet other embodiments, all cysteinyl residues in the polypeptides (other than the conjugatable moiety if it is a cysteine residue) are changed to serinyl residues. In other embodiments, the multimerization domain comprises a glycine-serine linker sequence. In some embodiments, the conjugatable moiety is an amino acid, and in particular embodiments, a cysteinyl residue having a sulfhydryl group.

In particular embodiments, the polypeptide is or comprises any one of SEQ ID Nos. 6-13.

Exemplary embodiments of the present disclosure include the following numbered embodiments:
1) A polypeptide, comprising:
   a. an adenovirus fiber polypeptide shaft domain motif;
   b. a sequence that induces opening of a tumor tight junction;
   c. a multimerization domain; and
   d. a moiety for targeted conjugation.
2) A polypeptide, comprising:
   a. an adenovirus fiber polypeptide shaft domain motif;
   b. a sequence that induces opening of a tumor tight junction; and
   c. a multimerization domain comprising a conjugatable moiety.
3) The polypeptide of embodiments 1-2, wherein the sequence that induces opening of a tumor tight junction is a sequence that binds desmoglein-2.
4) The polypeptide of embodiments 1-3, wherein the shaft domain motif is selected from the group consisting of an Ad3 fiber polypeptide shaft domain motif, an Ad7 fiber polypeptide shaft domain motif, an Ad11 fiber polypeptide shaft domain motif, an Ad 14 fiber polypeptide shaft domain motif, an Ad14a fiber polypeptide shaft domain motif, and combinations thereof.
5) The polypeptide of embodiment 4, comprising one or more shaft domain motifs having an amino acid sequence selected from the group consisting of SEQ ID NOS: 1-5.
6) The polypeptide of any one of embodiments 3-5, wherein the desmoglein 2 binding sequence is an adenovirus knob sequence derived from Ad3, Ad7, Ad11, Ad14, or Ad14a; wherein the desmoglein 2 binding sequence is modified to change or remove amino acids that could compete for conjugation with the conjugatable moiety.
7) The polypeptide of embodiments 1-6, wherein all cysteinyl residues in the polypeptide other than the conjugatable moiety are changed to serinyl residues.
8) The polypeptide embodiments 1-7, wherein the conjugatable moiety comprises an amino acid residue that is capable of covalent conjugation.
9) The polypeptide of embodiments 1-8, wherein the multimerization domain comprises a glycine-serine linker sequence.
10) The polypeptide of embodiments 8-9, wherein the amino acid capable of covalent conjugation is a cysteinyl residue.
11) The polypeptide of embodiment 10, wherein the cysteinyl residue used for conjugation also promotes multimerization.
12) The polypeptide of embodiment 1 comprising a sequence selected from:

a.
(SEQ ID NO: 6)
RGSHHHHHHGSKNSIALKNNTLWTGPKPEANSIIEYGKQNPDSKLTLI
LVKNGGIVNGYVTLMGASDYVNTLFKNKNVSINVELYFDATGHILPDS
SSLKTDLELKYKQTADFSARGFMPSTTAYPFDLPNAGTHNENYIFGQS
YYKASDGALFPLEVTVMLNKRLPDSRTSYVMTFLWSLNAGLAPETTQA
TLITSPFTFSYIREDDGGGSGGGSGGGSC;
b.
(SEQ ID NO: 7)
RGSHHHHHHGSKNSIALKNNTLWTGPKPEANSIIEYGKQNPDSKLTL
ILVKNGGIVNGYVTLMGASDYVNTLFKNKNVSINVELYFDATGHILP
DSSSLKTDLELKYKQTADFSARGFMPSTTAYPFDLPNAGTHNENFIF
GQSYYKASDGALFPLEVTVMLNKRLPDSRTSYVMTFLWSLNAGLAPE
TTQATLITSPFTFSYIREDDGGGSGGGSGGGSC;
C.
(SEQ ID NO: 8)
RGSHHHHHHGSKNSIALKNNTLWTGPKPEANSIIEYGKQNPDSKLTL
ILVKNGGIVNGYVTLMGASDYVNTLFKNKNVSINVELYFDATGHILP
DSSSLKTDLELKYKQTADFSARGFMPSTTAYPFDLPNAGTHNENYIF
GQSYYKASDGALFPLEVTVMLNKRLPDSRTSYVMTFLWSLSAGLAPE
TTQATLITSPFTFSYIREDDGGGSGGGSGGGSC;
d.
(SEQ ID NO: 9)
RGSHHHHHHGSKNSIALKNNTLWTGPKPEANSIIEYGKQNPDSKLTLI
LVKNGGIVNGYVTLMGASDYVNTLFKNKNVSINVELYFDATGHILPDS
SSLKTDLELKYKQTADFSARGFMPSTTAYPFDLPNAGTHNENFIFGQS
YYKASDGALFPLEVTVMLNKRLPDSRTSYVMTFLWSLSAGLAPETTQA
TLITSPFTFSYIREDDGGGSGGGSGGGSC;
e.
(SEQ ID NO: 10)
RGSKNSIALKNNTLWTGPKPEANSIIEYGKQNPDSKLTULVKNGGIVN
GYVTLMGASDYVNTLFKNKNVSINVELYFDATGHILPDSSSLKTDLEL
KYKQTADFSARGFMPSTTAYPFDLPNAGTHNENYIFGQSYYKASDGAL
FPLEVTVMLNKRLPDSRTSYVMTFLWSLNAGLAPETTQATLITSPFTF
SYIREDDGGGSGGGSGGGSC;
f.
(SEQ ID NO: 11)
RGSKNSIALKNNTLWTGPKPEANSIIEYGKQNPDSKLTLILVKNGGIVN
GYVTLMGASDYVNTLFKNKNVSINVELYFDATGHILPDSSSLKTDLELK
YKQTADFSARGFMPSTTAYPFDLPNAGTHNENFIFGQSYYKASDGALFP
LEVTVMLNKRLPDSRTSYVMTFLWSLNAGLAPETTQATLITSPFTFSYI
REDDGGGSGGGSGGGSC;
g.
(SEQ ID NO: 12)
RGSKNSIALKNNTLWTGPKPEANSIIEYGKQNPDSKLTLILVKNGGIV
NGYVTLMGASDYVNTLFKNKNVSINVELYFDATGHILPDSSSLKTDLE
LKYKQTADFSARGFMPSTTAYPFDLPNAGTHNENYIFGQSYYKASDGA
LFPLEVTVMLNKRLPDSRTSYVMTFLWSLSAGLAPETTQATLITSPFT
FSYIREDDGGGSGGGSGGGSC;
or
h.
(SEQ ID NO: 13)
RGSKNSIALKNNTLWTGPKPEANSIIEYGKQNPDSKLTULVKNGGIVN
GYVTLMGASDYVNTLFKNKNVSINVELYFDATGHILPDSSSLKTDLEL
KYKQTADFSARGFMPSTTAYPFDLPNAGTHNENFIFGQSYYKASDGAL
FPLEVTVMLNKRLPDSRTSYVMTFLWSLSAGLAPETTQATLITSPFTF
SYIREDDGGGSGGGSGGGSC.

13) The polypeptide of embodiment 1 wherein the multimerization domain functions to conjugates polypeptides to form a multimer.
14) The polypeptide of any one of embodiments 1-13, further comprising one or more compounds conjugated to the polypeptide.
15) The polypeptide of embodiment 14, wherein the one or more compounds are selected from the group consisting of therapeutics, diagnostics and imaging agents.
16) The polypeptide of embodiment 15, wherein the one or more compounds comprise at least one therapeutic, wherein the therapeutic is selected from the group consisting of antibodies, immunoconjugates, immune stimulators, CAR T-cells, nanoparticles, chemotherapeutics, radioactive particles, viruses, vaccines, cellular immunotherapy therapeutics, gene therapy constructs, nucleic acid therapeutics and combinations thereof.
17) An isolated nucleic acid encoding the polypeptide of any one of embodiments 1-12.
18) A recombinant expression vector comprising the isolated nucleic acid of embodiment 17.
19) A host cell comprising the recombinant expression vector of embodiment 18.
20) A pharmaceutical composition, comprising a polypeptide according to any of embodiments 1-16, and a pharmaceutically acceptable carrier.
21) A method for enhancing therapeutic treatment, or diagnosis of a disorder associated with epithelial tissue, and/or imaging epithelial tissues, comprising administering to a subject in need thereof:
   a. a therapeutic for treatment of the disorder, a diagnostic, or an imaging agent; and
   b. the pharmaceutical composition of embodiment 20, in an amount sufficient to enhance efficacy of the therapeutic, diagnostic, and imaging agent.
22) The method of embodiment 19, wherein the disorder associated with human tissue is selected from the group consisting of solid tumors, irritable bowel syndrome, inflammatory bowel disorder, Crohn's disease, ulcerative colitis, constipation, gatroesophageal reflux disease, Barrett's esophagus, chronic obstructive pulmonary disease, asthma, bronchitis, pulmonary emphysema, cystic fibrosis, interstitial lung disease, pneumonia, primary pulmonary hypertension, pulmonary embolism, pulmonary sarcoidosis, tuberculosis, pancreatitis, pancreatic duct disorders, bile duct obstruction, cholecystitis, choledocholithiasis, brain disorders, psoriasis, dermatitis, glomerulonephritis, hepatitis, diabetes, thyroid disorders, cellulitis, infection, pyelonephritis, multiple sclerosis, transplant rejection and gallstones.
23) The method of embodiment 22, wherein the disorder associated with epithelial tissue is a solid tumor.
24) The method of embodiment 23 wherein the solid tumor is selected from the group consisting of breast tumors, lung tumors, colon tumors, rectal tumors, stomach tumors, prostate tumors, ovarian tumors, uterine tumors, skin tumors, endocrine tumors, cervical tumors, kidney tumors, melanomas, pancreatic tumors, liver tumors, brain tumors, head and neck tumors, nasopharyngeal tumors, gastric tumors, squamous cell carcinomas, adenocarcinomas, bladder tumors and esophageal tumors.
25) The method of any one of embodiments 21-24 wherein one or more compounds comprises at least one therapeutic, wherein the therapeutic is selected from the group consisting of antibodies, immunoconjugates, immune stimulators, viruses, nanoparticles, chemotherapeutics, radioactive particle, vaccines, cellular immunotherapy therapeutics, gene therapy constructs, nucleic acid therapeutics and combinations thereof.
26) The method of any one of embodiments 21-24, wherein the therapeutic comprises a chemotherapeutic or a monoclonal antibody.
27) The method of any one of embodiments 21-24, wherein the therapeutic comprises an anti-tumor monoclonal antibody.
28) The method of embodiment 27, wherein the anti-tumor monoclonal antibody comprises an antibody selected from the group consisting of trastuzumab, cetuximab, pertuzumab, apomab, conatumumab, lexatumumab, bevacizumab, bevacizumab, denosumab, zanolimumab, lintuzumab, edrecolomab, rituximab, ticilimumab, tositumomab, alemtuzumab, epratuzumab, mitumomab, gemtuzumab ozogamicin, oregovomab, pemtumomab daclizumab, panitumumab, catumaxomab, ofatumumab and ibritumomab.
29) The method of any one of embodiments 21-27, wherein the disorder associated with epithelial tissue comprises a Her-2 positive tumor.
30) A method for improving delivery of a compound to an epithelial tissue, comprising contacting the epithelial tissue with
   a. one or more compounds to be delivered to the epithelial tissue; and
   b. a conjugated composition of any one of embodiments 1-16, or functional equivalent thereof, or the pharmaceutical composition of embodiment 20, sufficient to direct delivery of the one or more compounds to the epithelial tissue.
31) The method of embodiment 30, wherein the one or more compounds comprises a diagnostic or an imaging agent.
32) The method of embodiment 30 or 31, wherein the epithelial tissue comprises a solid tumor.
33) The method of embodiment 30, wherein the solid tumor is selected from the group consisting of breast tumors, lung tumors, colon tumors, rectal tumors, stomach tumors, prostate tumors, ovarian tumors, uterine tumors, skin tumors, endocrine tumors, cervical tumors, kidney tumors, melanomas, pancreatic tumors, liver tumors, brain tumors, head and neck tumors, nasopharyngeal tumors, gastric tumors, squamous cell carcinomas, adenocarcinomas, bladder tumors, and esophageal tumors.
34) A method for improving delivery of a substance to a tissue expressing desmoglein 2 (DSG2), comprising contacting the tissue expressing DSG2 with
   a. one or more compound to be delivered to the tissue; and
   b. linked to an amount of the recombinant protein of any one of embodiments 1-16, or functional equivalent thereof, or the pharmaceutical composition of embodiment 20, in an amount sufficient to enhance delivery of the one or more compounds to the tissue.

In this Brief Summary and the Detailed Description and the Claims, reference to the moiety for targeted conjugation may optionally be replaced by the term conjugatable moiety. The term moiety for targeted conjugation emphasizes that the conjugatable moiety is present in a polypeptide solely to function as a reaction site for a conjugation reaction, rather than to impart, e.g., any biological activity or necessary structure to the polypeptide, which may be relevant when the conjugatable moiety is an amino acid residue.

This Brief Summary has been provided to introduce certain concepts in a simplified form that are further described in detail below in the Detailed Description. The details of one or more embodiments are set forth in the Description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

FIGS. 1Aa, 1Ab, 1B, 1C and 1D collectively show the sequence and structure, of JO-1 and JOC-x. FIG. 1Aa shows the amino acid sequence of JO-1 (SEQ ID No: 14) (upper sequence) with the HIS tag (HHHHHH) residues in bold type, the dimerization domain highlighted in bold grey type, and the Ad3 knob highlighted in grey. The affinity-enhancing JO4 mutation (marked V239D in the figure), as well as the two internal cysteines at aa80 and aa 192 are shown in bold type with larger fonts. The H-I loop is underlined. In dimerization domain-deleted mutants, the residues deleted are shown by the dashed box. Cysteinyl residues were inserted at locations shown by black arrows. In clones containing a C-terminal cysteinyl residue, a flexible glycine-serine linker [(G₃S)₃; shown in grey italics)] was added to the C-terminal portion of the knob domain. FIG. 1Ab shows the sequence for JOC-x (SEQ ID NO: 6), following the convention used for FIG. 1Aa. FIG. 1B represents side view of the JO-4 trimer structure indicating the location of the N- and C-termini by solid grey or dashed black arrows, respectively. FIG. 1C shows a top down view of the trimer with grey arrows highlighting the DSG2 binding loop. The grey shaded oval approximates one JO-1 monomer. FIG. 1D shows a simple schematic of the parental JO-4 protein and ten JO-derivatives. The grey “C” characters indicate approximate locations of internal (JO-4 and GB1) or newly introduced (all other GBs) cysteinyl residues. Clones marked “V/D” indicate that versions of these clones were made with or without the affinity-enhancing V239D mutation.

FIGS. 2A, 2B, 2C, and 2D depict production and characterization of JO-1-derived proteins. FIG. 2A shows the boiled and reduced analysis of the GB panel on 4-20% SDS-PAGE gradient gels. FIG. 2B shows the non-reduced and un-boiled analysis of the same panel with the multimeric forms indicated to the right of the gel in panels a, b, and c whereby M=monomers, T=trimers, T-D=trimer-dimers, and Agg.=aggregates or multimers. FIG. 2C shows a DSG2 binding Western of the GB proteins in which all GB bound with the exception of GB5 and GB9. FIG. 2D shows GB protein-mediated viral inhibition measured by the VIA assay. In this graph, proteins containing a dimerization domain are shown in solid lines, while the dimerization deleted clones are shown with a dashed line. The IC₅₀ values for each of the JO-derivatives are shown in Table 1.

FIGS. 3A, and 3B show DSG2 shedding induced by junction openers. FIG. 3A shows the raw kinetics of DSG2 shedding throughout the course of the 48-hour assay. Note the baseline shedding of DSG2 in the PBS (control) treated cells. FIG. 3B shows the DSG2 shedding of JO-derivatives following subtraction of the DSG2 shedding in the PBS treated control.

FIGS. 4A, 4B, and 4C show the size exclusion chromatography (SEC) analysis of protein multimeric states for JO4 (FIG. 4A), GB3 (FIG. 4B), and GB7 (FIG. 4C). Approximate sizes of the proteins were determined by multi-angle light scattering (MALS) analysis. The solid arrows point to the A280 absorbance for the oxidized protein while the dashed arrows point to the A280 profile of the reduced protein. The y-axis represents the approximate fraction of each peak while the x-axis shows the elution time in minutes. The grey shaded boxes indicate the approximate retention time for (Agg.) aggregates, (Multi.) multimers, (T-D) trimer-dimers, (T) trimers, or (M) monomers based on MALS analysis. Numbers indicate the major peaks that were observed and were therefore chosen for MALS analysis. The sizes and relative percentages of each peak are shown in Table 2.

FIGS. 5A, 5B, and 5C show TEM images of protein aggregation and trimer-dimer formations. FIG. 5A shows the uranyl acetate negative staining of protein for JO4 (left), GB3 (middle), and GB7 (right). The white arrows indicate aggregates which appear nearly 50 nM in diameter according to the size bar at the lower left of the figure. FIG. 5B shows fine resolution imaging white particles seen in the GB7 image in panel A. These structures show the typical barbell structure assumed to be the trimer-dimer. FIG. 5C shows the space filling model of the trimer-dimer of JO-4 created in VMD using the known x-ray crystallographic coordinates of JO-4. The approximate sizes of the trimer-dimers seen in panel b appear to be consistent size of the structure modeled in VMD.

FIGS. 6A, 6B, and 6C show the viral inhibition curves mediated by reduced and non-reduced JO4 (FIG. 6A), GB3 (FIG. 6B), and GB7 (JOC-x) (FIG. 6C). In each blot, black lines show the VIA curves for the BSA control, grey dashed lines with solid diamond symbols show the curves for reduced proteins, and solid grey lines with open diamond symbols show the curves for the oxidized proteins. Table 3 reports the IC₅₀ values for the oxidized and reduced forms of each protein.

FIG. 7 shows results in a mouse model of lung cancer when JO-4 and affinity enhanced JOC-x are injected with the antitumoral therapy paclitaxel. In the A549 lung cancer model, paclitaxel alone (grey line, square symbols) does not affect outcomes, however, using either JO-4 (dark grey line, asterisk symbols) or JOC-x (lighter grey line, triangle symbols) tumor growth is significantly slowed compared with untreated mice (black line, black diamond symbols). Moreover, the combination of JOC-x with paclitaxel was just as effective as the JO-4 paclitaxel combination.

FIGS. 8A and 8B show a structure of pre-formed, sulfhydryl surface-reactive, pegylated, doxorubicin liposomes (e.g., Doxosome™ doxorubicin liposome, available from Encapsula NanoScience, Brentwood Tenn., USA, doxosome.com/product-tag/maleimide) (FIG. 8A) and one of five arms of the liposome depicting the coupling of JOC-x to the lipid moiety (FIG. 8B).

FIG. 9 shows the viral inhibition mediated by JOC-x alone (grey solid line, grey triangles) or following conjugation to PEG2- (dark grey dashed line, grey squares) or PEG11-biotin moieties (black dotted line, black triangles).

FIG. 10 shows viral inhibition curves for the JOC-DOTA (dark grey dashed line; dark grey triangles), JOC-DOTA (EU) (black dotted line; black squares), or JOC-x alone (grey solid line; grey triangles). The DOTA conjugates maintained their ability to bind DSG2 using the viral inhibition assay with IC₅₀ values of 0.055, 0.131, and 0.254 μg/mL for JOC-x, JOC-DOTA, and JOC-DOTA (EU), respectively.

FIG. 11 shows viral inhibition curves for the JOC-poly(I:C) conjugate (black dashed line; black circles) compared to JOC-x alone (grey solid line; grey triangles) or JOC-x+poly(I:C) (grey dotted line; grey diamonds). The JOC-poly(I:C) conjugates maintained their ability to bind DSG2 and inhibit viral entry, although an approximately 6.3-fold lower IC₅₀ of the conjugate relative to the unconjugated protein (0.203 vs 0.032 μg/mL, respectively) was observed.

FIG. 12 is a bar graph demonstrating the potency of a JOC-poly(I:C) conjugate to trigger innate signaling and activation through viral RNA sensors (TLR3 and MDA5) utilizing an in vitro HEK-Blue human TLR3 reporter cell line. Poly(I:C) was conjugated to JOC-x using the heterobifunctional crosslinker SMPB [succinimidyl 4-(p-maleimidophenyl) butyrate] linking the free cysteine on JOC-x to the amino terminus of poly(I:C). JOC-poly(I:C) conjugates (right-most bars) were able to bind TLR3 and induce signaling at significantly lower protein concentrations compared to poly(I:C) alone (left-most bars) or a mixture of JOC-x and poly(I:C) (middle bars). Each one was tested at concentrations from 20 μg/mL to 0.00128 μg/mL.

### DETAILED DESCRIPTION

The present disclosure provides polypeptides, particularly polypeptides which may open a tumor tight junction, e.g., desmoglein 2 binding proteins, where the polypeptides have, and may be modified to have, a moiety for targeted conjugation, e.g., a conjugatable moiety. When the moiety for targeted conjugation is an amino acid residue, the moiety for targeted conjugation is distinct from the amino acid residues that comprise the structural and biologically-functional aspects of the polypeptide. In other words, when the moiety for targeted conjugation is an amino acid residue, the moiety for targeted conjugation is not one of the amino acid residues that contribute to the biological function of the polypeptide, but instead it is a separate amino acid residue that has been included in the polypeptide for the sole purpose of allowing the polypeptide to be conjugated, e.g., conjugated to a compound as described herein. The polypeptide comprises an adenovirus fiber shaft domain motif, a sequence that opens a tumor tight junction, a multimerization domain, and a conjugatable moiety. In some embodiments, the multimerization domain comprises the conjugatable moiety, which is capable of multimerizing (e.g., dimerize) the polypeptides. The proteins may also have other modifications, such as linkage with therapeutic molecules.

### A. Adenovirus Fiber Polypeptides

The disclosure provides a polypeptide that may open a tumor tight junction, which may be referred to as a polypeptide that opens a tumor tight junction. One element of the polypeptide is an adenovirus fiber polypeptide (herein called “Ad shaft domain”).

The Ad shaft domain is part of an adenovirus fiber polypeptide. The adenovirus virion is an icosahedron characterized by a fiber located at the base of each of the 12 vertices of the capsid. The fiber on the virion is a homotrimeric structure consisting of three individual fiber polypeptides. Each adenovirus fiber polypeptide consists of an N-terminal tail, which interacts with the penton base protein of the capsid and contains the signals necessary for transport of the protein to the cell nucleus; a shaft, which contains a number of repeating motifs around 15 amino acid residues long (“shaft domain motif”); and a C-terminal knob domain that contains the determinants for receptor binding (Hong and Engler, J Virol. 70:7071-7078 (1996)). All adenoviruses attach to their receptors through the knob structure on the end of the fiber. The fiber polypeptides spontaneously assemble into homotrimers, referred to as “fibers,” which are located on the outside of the adenovirus virion at the base of each of the twelve vertices of the capsid.

The polypeptides of the present disclosure may comprise one, or more than one, shaft domains and a knob domain (sequence that opens a tumor tight junction), but typically, do not include a tail domain from an Ad fiber polypeptide, and in one embodiment explicitly do not include a tail domain from an Ad fiber polypeptide. The shaft domain motif and the knob domain of the disclosed compositions may be derived from any adenovirus serotype that uses DSG2 as an epithelial cell receptor for viral binding. At a minimum, these serotypes include Ad3, Ad7, Ad11, Ad14, and Ad14a. As other Ad serotypes are identified, those of ordinary skill in the art can readily identify those that bind DSG2 using binding assays known in the art. For example, surface plasmon resonance (SPR) studies using sensors containing immobilized recombinant DSG2 can be used to determine if new Ad serotypes bind to DSG2, combined with DSG2 competition studies. Further exemplary studies, such as loss and gain of function analyses, are described in detail in WO 2011/156761.

The polypeptides of the disclosure may comprise multiple shaft domain motifs or knob domains, or multimerization domains. Where more than one element (i.e., shaft domain motif, DSG2-binding sequence, and multimerization domain) is present, each element can have an identical sequence, or differ in sequence, such as being derived from different adenoviruses.

Reference shaft domain motifs comprise the Ad3 shaft domain motif: NSIALKNNTL SEQ ID NO: 1; the Ad7 shaft domain motif: NSNNICINDNINTL SEQ ID NO: 2; the Ad5 shaft domain motif: GAITVGNKNNDKLTL (SEQ ID NO: 3); the Ad11 and Ad 14 shaft domain motif: NSNNICIDDNINTL (SEQ ID NO: 4); and the Ad35 shaft domain motif: GDICIKDSINTL (SEQ ID NO: 5).

The sequence of the shaft domain or shaft domain motifs may include mutations (substitutions, additions, deletions, chimeras, etc.) as long as the shaft domain or motifs retain or improve binding affinity to DSG2 and retain or improve formation of multimers (such as dimers) via the multimerization domain.

The sequence that induces opening of a tumor tight junction, which may also be referred to herein as a sequence that opens a tumor tight junction, such as a sequence that binds DSG2 (e.g., Ad knob domain), will generally be derived from an adenovirus knob sequence. Desmoglein 2 is a membrane glycoprotein of epithelial cells and promotes formation of tight junctions between cells. Many epithelial cancers are known to highly upregulate the production of DSG2 resulting in formation of a network of cellular “staples” reminiscent of poorly organized junctions that make tumors difficult to permeate by cells and treatments. The knob sequence targets tumors by binding to desmoglein 2, and triggers the transient opening of tumor tight junctions.

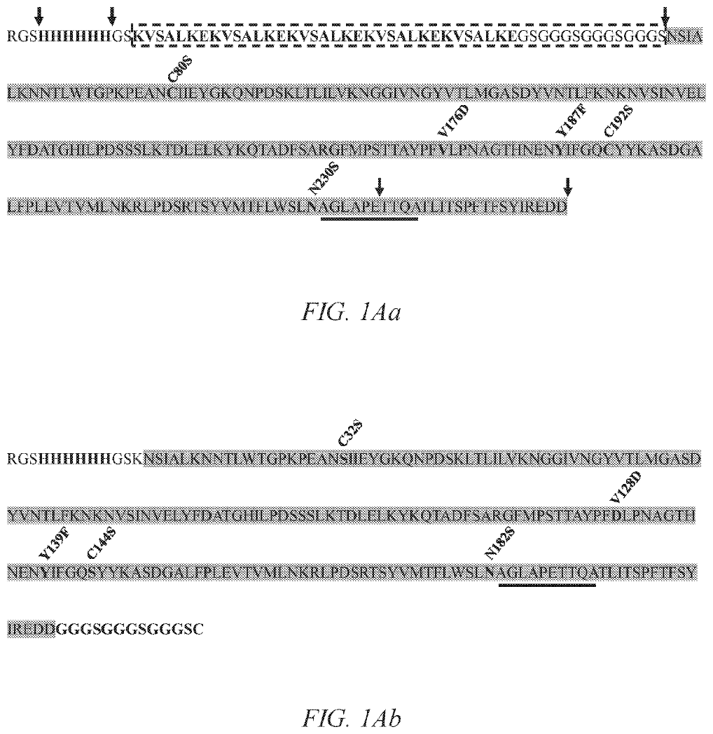

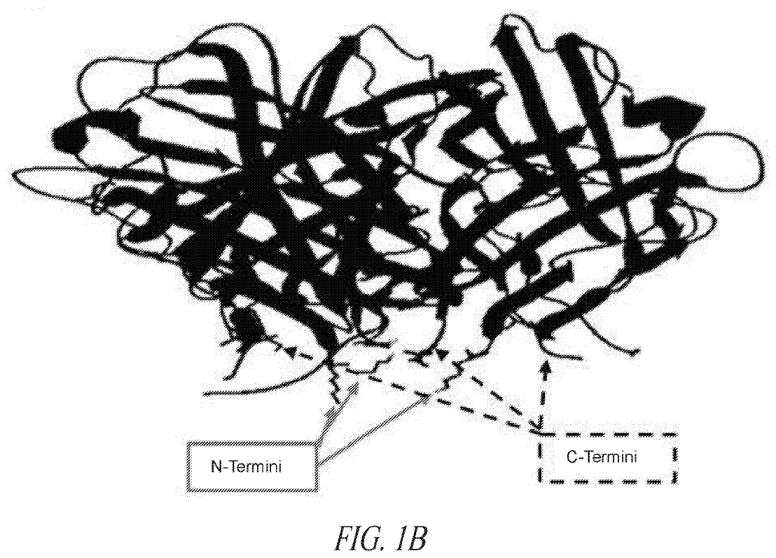

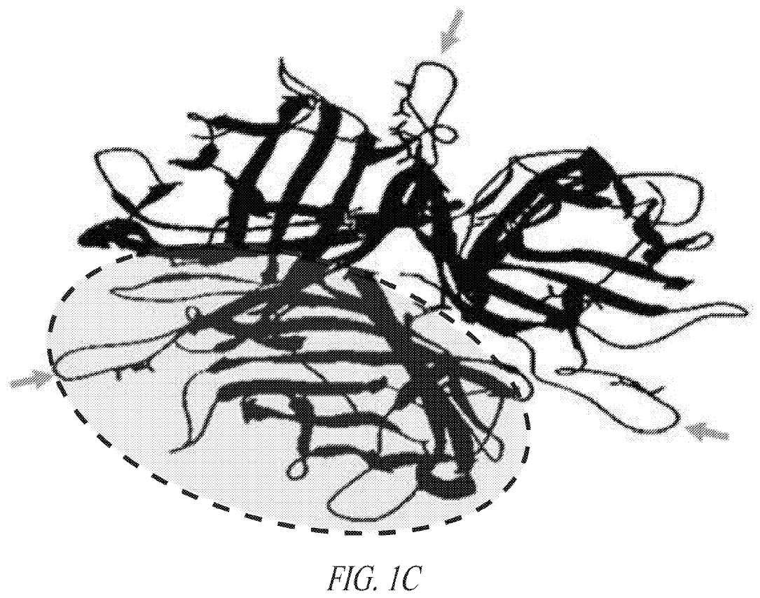

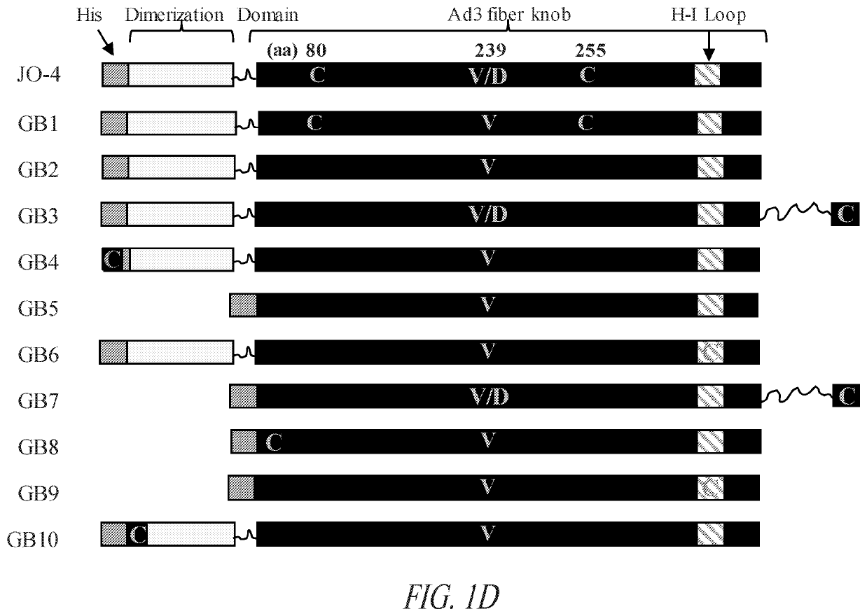

In some embodiments, the sequence of the knob domain is modified in order to e.g., enhance binding affinity to DSG2, reduce aggregation, increase production, and remove potential conjugatable amino acids. Exemplary modifications are disclosed in the examples. In JOC-x construct, several modifications were applied. In particular, the two cysteinyl residues at aa 32 and aa 144 were altered to serinyl residues (see FIG. 1A, b). In addition, aa 128 was changed from a Val to an Asp, which substantially increased binding avidity/affinity to DSG2. Additional affinity-enhancing mutations include Y139F and N182S. These, and other modifications, can be applied singly or in combinations.

The multimerization domain promotes the formation of trimers and/or trimer-dimers. Tumor tight junctions are opened best when the binding protein is in a dimer-trimer structure. Trimerization is likely mediated by self-assembly, and the presence of a dimerization domain is not needed for this process. Multimerization beyond this point is influenced by the inclusion of a multimerization sequence. A variety of sequences can mediate multimerization by either non-covalent (e.g., leucine zipper) means or covalent means. An exemplary covalent means is a Cys-Cys bonding. While an internal Cys can be used, adding a Cys at an N-terminal or C-terminal end appears to be a more efficient way. The disclosed polypeptides include a multimerization domain that comprises a conjugatable moiety (e.g., Cys), which is capable of both conjugating to an e.g. therapeutic molecule and mediating multimerization, and a separate multimerization domain and a conjugatable moiety, which is for conjugating a delivery molecule (e.g., therapeutic). In this latter case, the multimerization domain may promote formation of multimers either by covalent or noncovalent means. If covalent, typically the amino acid that reacts covalently will differ from the conjugatable moiety.

The disclosed polypeptide may also include a conjugatable moiety. In one embodiment, the conjugatable moiety will be capable of conjugating to a like moiety, e.g., the conjugatable moiety may be a sulfhydryl group from a cysteinyl residue which may react with itself to form a disulfide bond. In some embodiments, the conjugatable moiety will be capable of conjugating to a dissimilar moiety. As noted above, conjugation can be either covalent or non-covalent. Examples of conjugatable moieties include an amino acid with a reactive group, such as cysteines (Cys), a short run of amino acids, such as lysines (Lys), biotin or avidin/streptavidin, etc. Conjugation can be covalent (e.g., Cys-Cys), or non-covalent (e.g., ionic). When Cys is the conjugatable moiety, typically any internal Cys, whether or not they are bonded, are removed or changed to another amino acid. In one embodiment, the internal cysteinyl residues in the polypeptide are mutated to serinyl residues.

The polypeptide domain may additionally comprise other elements, e.g., chemicals, proteins, linker amino acids, etc. Typically, the additional elements will be located between the conjugatable moiety and the polypeptide of the composition. When linkers are used, the linkers may be of any length and sequence, although short sequences of flexible residues like glycine and serine that allow the polypeptide of the composition and the conjugatable moiety to move freely relative to one another are typically used. An exemplary linker sequence is GS (glycine-serine). A typical, flexible glycine serine linker is (G₃S)ₙ, wherein n=1 to 7. In one embodiment, the present disclosure provides a polypeptide comprising an (i.e., one or more) adenovirus fiber polypeptide shaft domain motifs, a sequence of amino acid residues that open a tumor tight junction, e.g., a sequence that binds desmoglein-2, a moiety that targets conjugation, e.g., a cysteinyl residue, and a linker such as (G₃S)ₙ, wherein n=1 to 7 that is bonded to both the moiety that targets conjugation and to the remainder of the polypeptide. In this way, the linker attaches a moiety that targets conjugation, or a conjugatable moiety, to the remainder of the polypeptide, so that a compound as disclosed herein can be conjugated to the remainder of the polypeptide.

The multimerization domain can be positioned at the N-terminus, the C-terminus, or internal to the polypeptide of the composition. It will most often be located at the N- or C-terminus.

The polypeptides of the compositions may comprise further domains, such as a domain for isolation of the polypeptide or a detection domain. An isolation domain can be added to facilitate purification/isolation of the polypeptide following, for example, recombinant polypeptide production. Any suitable isolation domain can be used, including but not limited to His, CBP, CYD (covalent yet dissociable NorpD peptide), Strep II, FLAG, HPC (heavy chain of protein C) peptide tags, GST and MBP affinity tags. A “detection domain” means one or more amino acid sequences that can be detected. Any suitable detection domain can be used, including but not limited to, inherently fluorescent proteins (e.g. Green-, Yellow-, or Red-Fluorescent Proteins and fluorescent proteins from nonbioluminescent Anthozoa species), cofactor-requiring fluorescent or luminescent proteins (e.g. phycobiliproteins or luciferases), and epitopes recognizable by specific antibodies or other specific natural or unnatural binding probes, including, but not limited to, dyes, enzyme cofactors and engineered binding molecules, which are fluorescently or luminescently labeled.

In embodiments, the polypeptides of the composition are in a multimeric form, such as a dimer, trimer, etc. In some embodiments, a multimer comprises a dimer formed by dimerization through the multimerization domains in each homotrimer (a polypeptide is a homotrimer through trimerization of the knob domain). In multimeric form (such as a dimer), the polypeptides of the disclosure comprise fiber multimers and can be used in the various methods of the invention discussed herein. Such multimers may comprise multimers of identical polypeptides, or may comprise multimers of different polypeptides.

The extent of multimerization can be determined according to methods well known to those of ordinary skill in the art. For example, multimerization of can be assessed by sedimentation in sucrose gradients, resistance to trypsin proteolysis, and electrophoretic mobility in polyacrylamide gels (Hong and Engler, J. of Virol. 70:7071-7078 (1996)). Regarding electrophoretic mobility, the fiber multimer is a very stable complex and will run at a molecular weight consistent with that of a multimer when the sample is not boiled and no reductant such as dithiothreitol (DTT) or beta-mercaptoethanol (2-ME) is added prior to SDS-PAGE. Upon boiling and reducing, however, the multimeric structure is disrupted and the protein subsequently runs at a size consistent with the protein monomer.

### B. Nucleic Acids Encoding the Polypeptides

In another aspect, the disclosure provides nucleic acids encoding the polypeptide of any aspect or embodiment of the invention. The nucleic acids may comprise RNA or DNA, and can be prepared and isolated using standard molecular biological techniques. The nucleic acids may comprise additional domains useful for promoting expression and/or purification of the encoded protein, including but not limited to polyA sequences, modified Kozak sequences, and sequences encoding epitope tags, export signals, and secretory signals, nuclear localization signals, and plasma membrane localization signals.

In a further aspect, the disclosure provides recombinant expression vectors comprising the nucleic acid of any aspect or embodiment of the invention operatively linked to a promoter. “Recombinant expression vector” includes vectors that operatively link a nucleic acid coding region or gene to any promoter capable of effecting expression of the gene product. The promoter sequence used to drive expression of the disclosed nucleic acids in a mammalian system may be constitutive (driven by any of a variety of promoters including, but not limited to, CMV, SV40, RSV, actin, EF) or inducible (driven by any of a number of inducible promoters including, but not limited to, tetracycline, ecdysone, steroid-responsive). The construction of expression vectors for use in transfecting prokaryotic cells is also well known in the art, and thus can be accomplished via standard techniques. The expression vector may be replicable in the host organisms and may comprise any other components as deemed appropriate for a given use, including but not limited to selection markers such as an antibiotic-resistance gene.

The disclosure also provides prokaryotic and eukaryotic host cells comprising the recombinant expression vectors disclosed herein. The cells can be transiently or stably transfected. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated-transfection. Techniques utilizing cultured cells transfected with expression vectors to produce quantities of polypeptides are well known in the art.

The polypeptides may be enriched or purified using any appropriate technique. They may be stored as monomers or multimers for future use.

### C. Formulations and Uses

The present disclosure provides pharmaceutical compositions, comprising a polypeptide of the present disclosure and a pharmaceutically acceptable carrier. Optionally, the polypeptide structure may be that of a multimer. The multimer can be, e.g., any of a dimer, trimer, dimer-trimer or higher order multimer. The polypeptide or multimer thereof may be conjugated to a therapeutic agent.

The pharmaceutical composition may further comprise one or more therapeutics for treating a disorder associated with epithelial tissue. The therapeutic may be an anti-tumor therapeutic and comprising a chemotherapeutic or anti-tumor monoclonal antibody (mAb). Many anti-tumor mAbs are known in the art and are being developed. These include trastuzumab, cetuximab, pertuzumab, apomab, conatumumab, lexatumumab, bevacizumab, bevacizumab, denosumab, zanolimumab, lintuzumab, edrecolomab, rituximab, ticilimumab, tositumomab, alemtuzumab, epratuzumab, mitumomab, gemtuzumab ozogamicin, oregovomab, pemtumomab daclizumab, panitumumab, catumaxomab, ofatumumab and ibritumomab3F8, Abagovomab, Abituzumab, Adecatumumab, Altumomab pentetate, Amatuximab, Anatumomab mafenatox, Arcitumomab, Ascrinvacumab, Bavituximab, Bectumomab, Bivatuzumab mertansine, Blinatumomab, Blontuvetmab (veterinary), Brentuximab vedotin, Brontictuzumab, Cantuzumab mertansine, Cantuzumab ravtansine, Capromab pendetide, Citatuzumab bogatox, Cixutumumab, Clivatuzumab tetraxetan, Dacetuzumab, Dalotuzumab, Daratumumab, Demcizumab, Denintuzumab mafodotin, Depatuxizumab mafodotin, Derlotuximab biotin, Detumomab, Dinutuximab, Drozitumab, Duligotumab, Dusigitumab, Ecromeximab, Elotuzumab, Emactuzumab, Emibetuzumab, Enfortumab vedotin, Enoblituzumab, Enoticumab, Ensituximab, Ertumaxomab, Etaracizumab, Farletuzumab, Ficlatuzumab, Figitumumab, Flanvotumab, Futuximab, Ganitumab, Girentuximab, Glembatumumab vedotin, lcrucumab, lgovomab, Imgatuzumab, Indatuximab ravtansine, Inotuzumab ozogamicin, Intetumumab, Iratumumab, Isatuximab, Labetuzumab, Lifastuzumab vedotin, Lorvotuzumab mertansine, Lucatumumab, Lumretuzumab, Mapatumumab, Margetuximab, Matuzumab, Milatuzumab, Minretumomab, Moxetumomab pasudotox, Nacolomab tafenatox, Naptumomab estafenatox, Narnatumab, Necitumumab, Nesvacumab, Nimotuzumab, Nofetumomab merpentan, Obinutuzumab, Ocaratuzumab, Olaratumab, Onartuzumab, Ontuxizumab, Oportuzumab monatox, Otlertuzumab, Parsatuzumab, Patritumab, Pinatuzumab vedotin, Pintumomab, Polatuzumab vedotin, Pritumumab, Racotumomab, Radretumab, Ramucirumab, Rilotumumab, Robatumumab, Satumomab pendetide, Seribantumab, Sibrotuzumab, Siltuximab, Simtuzumab, Sofituzumab vedotin, Solitomab, Tacatuzumab tetraxetan, Tamtuvetmab, Taplitumomab paptox, Tarextumab, Tenatumomab, Teprotumumab, Tigatuzumab, Tovetumab, Tucotuzumab celmoleukin, Ublituximab, Vandortuzumab vedotin, Vantictumab, Vanucizumab, Veltuzumab, Vesencumab, Vorsetuzumab mafodotin, Votumumab, and Zalutumumab

The pharmaceutically acceptable carrier is non-toxic, biocompatible and is selected so as not to detrimentally affect the biological activity of the multimers (and any other therapeutic agents combined therewith). Exemplary pharmaceutically acceptable carriers for peptides are described in U.S. Pat. No. 5,211,657. The compositions may be formulated into preparations in solid, semi-solid, gel, liquid or gaseous forms such as tablets, capsules, powders, granules, ointments, solutions, suppositories, inhalants, and injections, allowing for oral, parenteral, or surgical administration. Suitable carriers for parenteral delivery via injectable, infusion, or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids, such as oleic acid, find use in the preparation of injectables. The carrier and agent may be compounded as a liquid, suspension, polymerizable or non-polymerizable gel, paste, or salve. The carrier may also comprise a delivery vehicle to sustain (i.e., extend, delay, or regulate) the delivery of the agent(s) or to enhance the delivery, uptake, stability, or pharmacokinetics of the therapeutic agent(s). Examples of such delivery vehicles include microparticles, microspheres, nanospheres, or nanoparticles composed of proteins, liposomes, carbohydrates, synthetic organic compounds, inorganic compounds, polymeric or copolymeric hydrogels, and polymeric micelles. Suitable hydrogel and micelle delivery systems may be used.

For intrathecal (IT) or intracerebroventricular (ICV) delivery, appropriately sterile delivery systems (e.g., liquids; gels, suspensions, etc.) can be used to administer the compositions. For oral administration the compositions may be carried in an inert filler or diluent such as sucrose, cornstarch, or cellulose.

Pharmaceutical compositions may also include biocompatible excipients, such as dispersing or wetting agents, suspending agents, diluents, buffers, penetration enhancers, emulsifiers, binders, thickeners, flavoring agents (for oral administration). Exemplary formulations can be parenterally administered as injectable dosages of a solution or suspension of the multimer in a physiologically acceptable diluent with a pharmaceutical carrier that can be a sterile liquid such as water, oils, saline, glycerol, or ethanol. Additionally, auxiliary substances such as wetting or emulsifying agents, surfactants, pH buffering substances and the like can be present in compositions comprising modified polypeptides. Additional components of pharmaceutical compositions may include petroleum (such as of animal, vegetable, or synthetic origin), for example, soybean oil and mineral oil. In general, glycols such as propylene glycol or polyethylene glycol are preferred liquid carriers for injectable solutions.

The pharmaceutical composition can also be administered in the form of a depot injection or implant preparation that can be formulated in such a manner as to permit a sustained or pulsatile release of the multimers and other therapeutics (if present).

The pharmaceutical composition may also comprise (a) a lyoprotectant; (b) a surfactant; (c) a bulking agent; (d) a tonicity adjusting agent; (e) a stabilizer; (f) a preservative; and/or (g) a buffer. In some embodiments, the buffer in the pharmaceutical composition is a Tris buffer, a histidine buffer, a phosphate buffer, a citrate buffer, or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The pharmaceutical composition can be packaged in any suitable manner. In one embodiment, the pharmaceutical composition is packaged as a kit containing a container (such as a vial) of the disclosed compositions. The kit may further comprise, in the same or a separate container (such as a vial), a therapeutic, diagnostic, or imaging agent to be administered to a subject, together with the composition.

The present disclosure also provides kits comprising (a) one or more compositions, isolated nucleic acids, recombinant expression vectors, and/or host cells of the invention; and (b) instructions for its/their use in treating a disorder associated with epithelial tissue. The kits may further comprise a therapeutic for use in the methods of the present invention.

The present disclosure also provides methods for enhancing therapeutic treatment, diagnosis of a disorder associated with epithelial tissue, and imaging epithelial tissues, comprising administering to a subject in need thereof: (a) an amount of one or more therapeutics sufficient to treat the disorder, diagnostic sufficient to diagnose the disorder, and/or imaging agent sufficient to image the epithelial tissue; and (b) the disclosed compositions in an amount sufficient to enhance efficacy of the one or more therapeutics, diagnostics, and/or imaging agents.

In another embodiment, the polypeptide is combined with (such as conjugated to) one or more therapeutics for a disorder associated with epithelial tissue. Such conjugates can be used, for example, in the therapeutic methods disclosed herein. Methods for conjugating the polypeptides of the compositions to a therapeutic of interest, such as by covalent binding or chemical cross-linking, are well known to those of ordinary skill in the art. Any suitable therapeutic can be used to form a conjugate, including but not limited to tumor stroma degrading compounds (such as relaxin), alkylating agents, angiogenesis inhibitors, antibodies, antimetabolites, antimitotics, antiproliferatives, aurora kinase inhibitors, apoptosis promoters (for example, Bcl-xL, Bcl-w and Bfl-1) inhibitors, activators of death receptor pathway, Bcr-Abl kinase inhibitors, BiTE (Bi-Specific T cell Engager) antibodies, biologic response modifiers, cyclin-dependent kinase inhibitors, cell cycle inhibitors, cyclooxygenase-2 inhibitors, growth factor inhibitors, heat shock protein (HSP)-90 inhibitors, demethylating agents, histone deacetylase (HDAC) inhibitors, hormonal therapies, immunologicals, inhibitors of apoptosis proteins (IAPs) intercalating antibiotics, kinase inhibitors, mammalian target of rapamycin inhibitors, microRNA's mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors and the like.

In another embodiment, the therapeutic comprises a compound that binds to desmoglein-2; preferably a compound that binds to DSG2 and opens up tight junctions. The therapeutic may comprise radioactive particles for radiation therapy. Any suitable radioactive therapy or particle can be used as deemed appropriate by an attending physician, including but not limited to cobalt-60, copper-64, iodine-131, iridium-192, strontium-89, samarium-153, rhenium-186, yttrium-90, and lead-212. The therapeutic may be an anti-tumor therapeutic and comprise a chemotherapeutic or anti-tumor monoclonal antibody.

The imaging agent can be a fluorescent imaging agent, while diagnostic agents may comprise a compound that is a diagnostic marker for a particular epithelial disorder bound to the fluorescent imaging agent. A fluorescent imaging agent is any chemical moiety that has a detectable fluorescence signal. This imaging agent can be used alone or in combination with other imaging agents. The imaging agents can comprise a Magnetic Resonance Imaging (MRI) agent, while diagnostic agents may comprise a compound that is a diagnostic marker for a particular epithelial disorder bound to the MRI agent. A MRI agent is any chemical moiety that has a detectable magnetic resonance signal or that can influence (e.g., increase or shift) the magnetic resonance signal of another agent. This type of imaging agent can be used alone or in combination with other imaging agents. Other imaging agents include PET agents that can be prepared by incorporating an 18F or a chelator, such as DOTA, DOTATOC, DOTANOC, DOTA-TATE, DTPA, or NOTA, for 64Cu or 68Ga. Also, addition of a radionuclide can be used to facilitate SPECT imaging or delivery of a radiation dose, while diagnostic agents may comprise a compound that is a diagnostic marker for a particular epithelial disorder bound to the PET agent.

In other embodiments, the diagnostic agent is a marker gene that encodes proteins that are readily detectable when expressed in a cell (including, but not limited to, beta-galactosidase, green fluorescent protein, luciferase, and the like) and labeled nucleic acid probes (e.g., radiolabeled or fluorescently labeled probes). In some embodiments, covalent conjugation of diagnostic or imaging agents to the AdB-2/3 multimers provided herein is achieved according to a variety of conjugation processes. In other embodiments, the diagnostic agent is non-covalently associated with the disclosed compositions.

The methods of the invention have broad application for delivery of any therapeutic, diagnostic, imaging agent, or other compound to epithelial tissue comprising intercellular junctions where access to a target of interest can be limited, as DSG2 is widely expressed in epithelial cells. As used herein, a “disorder associated with epithelial tissue” is any disorder wherein therapeutic, diagnostic, or imaging agent administered to/across epithelial cells/epithelial tissue provides a clinical benefit to a patient, whether in improving therapeutic, diagnostic, and/or imaging efficacy. Such disorders include, but are not limited to, solid tumors (i.e. any tumor with epithelial cell junctions), gastrointestinal disorders (including, but not limited to, irritable bowel syndrome, inflammatory bowel disorder, Crohn's disease, ulcerative colitis, constipation, gastroesophageal reflux disease, Barrett's esophagus, etc.), skin diseases (including, but not limited to, psoriasis and dermatitis), lung disorders (including, but not limited to, chronic obstructive pulmonary disease, asthma, bronchitis, pulmonary emphysema, cystic fibrosis, interstitial lung disease, pneumonia, pancreatic duct disorders, brain disorders (ie: any brain disorder that could benefit from improved transport of drugs through the blood-brain barrier), primary pulmonary hypertension, pulmonary embolism, pulmonary sarcoidosis, tuberculosis, etc.), renal disorders, (including but not limited to glomerulonephritis), liver diseases (including but not limited to hepatitis), endocrine disorders (including but not limited to diabetes and thyroid disorders), pancreatic duct disorders (including but not limited to pancreatitis), and bile duct disorders (including but not limited to bile duct obstruction, cholecystitis, choledocholithiasis, gallstones, etc.) and infections of epithelial tissues (including but not limited to cellulitis, pneumonia, hepatitis, and pyelonephritis). In one preferred embodiment, the disorder associated with epithelial tissue comprises a solid tumor, including but not limited to breast tumors, lung tumors, colon tumors, rectal tumors, skin tumors, endocrine tumors, stomach tumors, prostate tumors, ovarian tumors, uterine tumors, cervical tumors, kidney tumors, melanomas, pancreatic tumors, liver tumors, brain tumors, head and neck tumors, nasopharyngeal tumors, gastric tumors, squamous cell carcinomas, adenocarcinomas, bladder tumors, and esophageal tumors. As will be understood by those of skill in the art, such tumors include primary tumors, tumors that are locally invasive, as well as tumors that have metastasized.

As used herein, “enhancing efficacy” means any increase in therapeutic, diagnostic, and/or imaging efficacy over what would be seen using the therapeutic, diagnostic, and/or imaging agent alone. For example, measurements of therapeutic efficacy will vary depending on the disorder being treated, but are readily identified by an attending physician. For example, such increases in efficacy include, but are not limited to, increasing one or more of the following relative to treatment with the therapeutic alone: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s). In one non-limiting example, treating a solid tumor provides an ability to induce egress of tumor receptors from the basolateral side of epithelial cells to enable improved access and killing of the tumor.

For cancer, there are standards for defining tumor response and standard methods of measuring response. These include tumor response, which is determined by monitoring the change in tumor size or a serum marker of disease. A partial response is more than a 50% reduction in the tumor, while a complete response is defined as complete disappearance of the tumor. Methods used to measure tumors are well known to physicians and include physical examination, radiological testing such as CT scans, MRI, PET scans, X-rays as well as serum markers such as prostate specific antigen, which is used to monitor prostate cancer. Other measures of therapeutic efficacy of cancer treatment include measurements of time to progression, progression-free survival and overall survival.

Improved diagnostic efficacy includes any improvement in efficacy compared to administration of the diagnostic alone, including but not limited to, increasing specificity and/or sensitivity of the diagnostic test. Improved imaging efficacy includes any improvement in efficacy compared to administration of the imaging agent alone, including but not limited to specificity, sensitivity, reproducibility, contrast enhancement, detection of smaller sites of disease, more accurate delineation of disease, such as size and shape of diseases, such as tumors, abscesses, etc.

In various embodiments, the increase in efficacy is a 5%, 10%, 15%, 20%, 25%, 50%, 75%, 100%, or greater benefit compared to efficacy with the therapeutic, diagnostic, and/or imaging agent alone across a patient population.

Any suitable subject can be treated using the methods of the invention, including human and animal subjects.

In various embodiments, the therapeutic is selected from the group consisting of alkylating agents, angiogenesis inhibitors, antibodies, antimetabolites, antimitotics, antiproliferatives, aurora kinase inhibitors, apoptosis promoters (for example, Bcl-xL, Bcl-w and Bfl-1) inhibitors, activators of death receptor pathway, Bcr-Abl kinase inhibitors, BiTE (Bi-Specific T cell Engager) antibodies, biologic response modifiers, cyclin-dependent kinase inhibitors, cell cycle inhibitors, cyclooxygenase-2 inhibitors, growth factor inhibitors, heat shock protein (HSP)-90 inhibitors, demethylating agents, histone deacetylase (HDAC) inhibitors, hormonal therapies, immunologicals, inhibitors of apoptosis proteins (IAPs) intercalating antibiotics, kinase inhibitors, mammalian target of rapamycin inhibitors, microRNA's mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors and the like.

In one embodiment, the disorder comprises a Her-2 positive tumor, and the method comprises co-administering the compositions disclosed herein together with suitable monoclonal antibody therapy (e.g., trastuzumab), alone or in combination with a chemotherapeutic, radiation, or combinations thereof. In some embodiments, the Her-2 positive tumor is selected from the group consisting of a breast tumor, a gastric tumor, a colon tumor, and an ovarian tumor. In embodiments, the method is carried out on patients who have not responded adequately to trastuzumab, such as by lack of tumor remission, by tumor relapse, or by development of resistance to trastuzumab. The methods of these embodiments can also be used to help reduce the dosage of trastuzumab required to obtain therapeutic efficacy, and can thus serve to limit side effects (such as trastuzumab-associated cardiotoxicity).

In another embodiment, the disorder comprises an EGFR-positive tumor, and the method comprises co-administering the compositions disclosed herein together with suitable monoclonal antibody therapy (e.g., cetuximab), alone or in combination with a chemotherapeutic, radiation, or combinations thereof. In a further preferred embodiment, the monoclonal antibody is cetuximab. The EGFR-positive tumor may be a lung tumor, a colon tumor, a breast tumor, a rectal tumor, a head and neck tumor, and a pancreatic tumor. In embodiments, the method is carried out on patients who have not responded adequately to cetuximab, such as by lack of tumor remission, by tumor relapse, or by development of resistance to cetuximab. The methods of these embodiments can also be used to help reduce the dosage of cetuximab required to obtain therapeutic efficacy, and can thus serve to limit side effects (such as acne-like rashes that often occur during cetuximab therapy).

In similar fashion, the disorder comprises an epithelial tumor, and the method comprises co-administering a composition disclosed herein together with a vascular endothelial growth factor (VEGF) inhibitor, alone or in combination with other chemotherapeutic, radiation, or combinations thereof. Any suitable VEGF inhibitor can be used, including but not limited to bevacizumab. In further embodiments, the methods involving solid tumors further comprise administering a compound capable of degrading tumor stroma proteins. Any suitable compound for degrading tumor stroma proteins can be used, including but not limited to relaxin, collagenase, trypsin, dispase, MMP (metalloproteinase)-1, and MMP8. Delivery of such compounds can be by any suitable mechanism, including gene therapy, separate administration with the disclosed compositions, or administration as a conjugate.

In a further embodiment that can be combined with any embodiment or combination of embodiments herein, the methods further comprise administering the AdB-2/3 multimer in combination with other junction openers. As used herein, a “junction opener” is a compound capable of transiently opening intercellular junctions. Any suitable junction openers can be used. In one non-limiting embodiment, the junction opener comprises Zona occludens toxin (Zot), a Vibrio cholerae (V. cholerae)-produced toxin that possess the ability to reversibly alter intestinal epithelial junctions, allowing the passage of macromolecules through mucosal barriers (Fasano et al. (1991) Proc Natl Acad Sci USA 88: 5242-5246)]. A Zot-derived hexapeptide (AT-1001) has been developed. In another embodiment, Clostridium perfringens enterotoxin removes claudins-3 and -4 from the tight junctions to facilitate bacterial invasion (Sonoda N, et al. (1999) J Cell Biol 147: 195-2041. In a further embodiment, oncoproteins encoded by human Ad, HPV, HTLV-1 can transiently open epithelial junctions by mislocalizing the junction protein ZO-1 (Latorre I J, et al. (2005) J Cell Sci 118: 4283-4293). In other embodiments, several human viruses engage tight junction or other cell junction molecules to achieve entry into epithelial cells. Among these viruses are hepatitis C virus (Evans M J, et al. (2007) Nature 446: 801-805), reovirus (Barton E S, et al. (2001) Cell 104: 441-451), and herpes simplex virus (Geraghty R J, et al. (1998) Science 280: 1618-1620).

In another embodiment, the therapeutic is an inhaled therapeutic. Any suitable inhaled therapeutic can be used in the methods of the invention. In various non-limiting embodiments, the inhaled therapeutic is selected from the group consisting of corticosteroids, bronchodilators, beta agonists, anticholinergics, albuterol (PROVENTIL®; VENOLIN®; ACCUNEB®; PROAIR®), levalbuterol (XOPENEX®), pirbutrol (MAXAIR®), ipratropium bromide (ATROVENT®), beclomethasone, budesonide, flunisolide (AEROBID®), fluticasone, triamcinolone acetonide, fluticasone (a corticosteroid) and salmeterol (ADVAIR®), formotorol (a long-acting, beta-agonist bronchodilator) and budesonide (a corticosteroid) (SYMICORT®), albuterol (a beta agonist) and ipratropium (COMBIVENT®; an anticholinergic) budesonide (PULMICORT RESPULES®), and tiopropium (SPIRIVA®; an anticholinergic bronchodilator).

Administering the compositions disclosed herein can improve delivery of a substance to an epithelial tissue, such as an epithelial tumor. The method comprises contacting epithelial tissue with (a) one or more compound to be delivered to the epithelial tissue, and (b) an amount sufficient of a disclosed composition to enhance delivery of the one or more compounds to the epithelial tissue. The compounds may be any suitable compound such as those described herein, including an imaging agent, therapeutic agent, and diagnostic agent.

The present disclosure also provides methods for improving delivery of a substance cell or tissue expressing desmoglein 2 (DSG2), comprising contacting the cell or tissue expressing DSG2 with (a) one or more compound to be delivered to the cell or tissue; and (b) an amount sufficient of a disclosed composition to enhance delivery of the one or more compounds to the tissue. Exemplary tissue types expressing DSG2 include, but are not limited to epithelial tissue, human platelets and granulocytes.

The present disclosure also provides methods for inducing an epithelial to mesenchymal transition (EMT) in a tissue, comprising contacting the epithelial tissue with an amount of a composition disclosed herein sufficient to induce EMT. EMT is a cellular transdifferentiation program where epithelial cells lose characteristics such as intercellular junctions and gain properties of mesenchymal cells.

In all of the aspects and embodiments of the disclosed methods, the therapeutic, diagnostic, and imaging agent can be administered together with the compositions of the disclosure or may be administered separately. When administered together, the agent and the composition may be conjugated, via any suitable covalent or non-covalent binding.

The compositions can be administered in any way deemed suitable by an attending physician, depending on whether a local or systemic mode of administration is most appropriate for the condition being treated. As used herein, the terms “systemic delivery” and “systemic administration” are intended to include, but are not limited to, oral and parenteral routes including intramuscular (IM), subcutaneous, intravenous (IV), intra-arterial, inhalational, sublingual, buccal, topical, transdermal, nasal, rectal, vaginal, and other routes of administration that effectively result in dispersal of the delivered agent to a single or multiple sites of intended therapeutic action.

The compositions and therapeutic may be systemically administered on a periodic basis at intervals determined to maintain a desired level of therapeutic effect. For example, administration by intravenous injection may be once per day, once per week, every two to four weeks or at less frequent intervals. The dosage regimen will be determined by the physician considering various factors that may influence the action of the combination of agents. These factors may include the extent of progress of the condition being treated, the patient's age, sex and weight, and other clinical factors. The dosages of the compositions and the therapeutic may vary as a function of the multimer or therapeutic being administered, as well as the presence and nature of any drug delivery vehicle (e.g., a sustained release delivery vehicle). In addition, the dosage quantity may be adjusted to account for variation in the frequency of administration and the pharmacokinetic behavior of the delivered agent(s). Dosages of approved therapeutics are readily identifiable by medical practitioners. The therapeutic may also be able to be administered at a reduced dose due to enhanced penetration into epithelial tissues.

In another aspect, methods are provided for treating a disorder associated with epithelial tissue, comprising administering to a subject in need thereof an amount of a composition disclosed herein sufficient to treat the disorder. No other therapeutic is delivered. In non-limiting embodiments, the monotherapy is used to treat a disorder selected from the group consisting of an AdB-2/3 viral infection, a solid tumor, or a disorder that can be treated using an AdenovirusB-2/3-based gene delivery vector. For example, in treating solid tumors, the method comprises improving access of immune system cells to the site of the disorder, such as by penetration (such as intratumoral penetration of pre-existing natural killer cells, T-cells or dendritic cells). The method can also be used to treat any of the disorders associated with epithelial cells discussed above that can benefit from improved access of cells of the immune system to the target epithelial cells.

The present disclosure also provides methods for identifying candidate compounds for treating a disorder associated with epithelial tissue, improving delivery of a substance to an epithelial tissue, improving delivery of a substance tissue expressing DSG2, inducing an EMT in a tissue, and treating an AdB-2/3 infection. The methods comprise (a) contacting a composition disclosed herein to DSG2 under conditions to promote multimer binding to DSG2, wherein the contacting is carried out in the presence of one or more test compounds; and (b) identifying positive test compounds that compete with the composition for binding to DSG2 compared to control. Positive test compounds are candidate compounds for transiently opening intracellular junctions through their interaction with DSG2. Follow-up assays to verify the ability of the compounds to transiently open intracellular junctions through their interaction with DSG2 can be carried out by any suitable methods.

The following are some exemplary embodiments of the present disclosure:
1) A composition that opens a tumor tight junction, comprising:
   a. one or more adenovirus fiber polypeptide shaft domain motifs; and
   b. a multimerization domain located either N′ or C′ of the fiber polypeptide shaft domain, wherein the multimerization domain comprises a conjugatable moiety.
2) The composition of embodiment 1, wherein each shaft domain motif is selected from the group consisting of an Ad3 fiber polypeptide shaft domain motif, an Ad7 fiber polypeptide shaft domain motif, an Ad11 fiber polypeptide shaft domain motif, an Ad 14 fiber polypeptide shaft domain motif, an Ad14a fiber polypeptide shaft domain motif, or any combinations thereof.
3) The composition of any one of embodiments 1 or 2, wherein the one or more shaft domain motifs comprise 1-22 shaft domain motifs.
4) The composition of any one of embodiments 1-3, wherein each shaft domain motif comprises an amino acid sequence selected from the group consisting of SEQ ID NOS: 1-5.
5) The composition of embodiment 1-4, wherein all naturally occurring cysteinyl residues in the adenovirus sequence are changed to serinyl residues.
6) The composition of embodiment 5, wherein all the natural cysteinyl residues at positions aa80 and 151 have been mutated to serinyl residues (C80S, C151S). 7) The composition of any one of embodiments 1-6 wherein the multimerization domain contains an amino acid residue that is capable of covalent conjugation.
8) The composition of embodiment 7 wherein the multimerization domain contains a glycine-serine linker sequence.
9) The composition of any one of embodiments 7-8 wherein the amino acid capable of covalent conjugation is a cysteinyl residue.
10) The composition of any one of embodiments 1-9, wherein the polypeptide sequence comprises any one of

a.
(SEQ ID NO: 6)
RGSHHHHHHGSKNSIALKNNTLWTGPKPEANSIIEYGKQNPDSKLTL
ILVKNGGIVNGYVTLMGASDYVNTLFKNKNVSINVELYFDATGHILP
DSSSLKTDLELKYKQTADFSARGFMPSTTAYPFDLPNAGTHNENYIF
GQSYYKASDGALFPLEVTVMLNKRLPDSRTSYVMTFLWSLSAGLAPE
TTQATLITSPFTFSYIREDDGGGSGGGSGGGSC;
b.
(SEQ ID NO: 7)
RGSHHHHHHGSKNSIALKNNTLWTGPKPEANSIIEYGKQNPDSKLTL
ILVKNGGIVNGYVTLMGASDYVNTLFKNKNVSINVELYFDATGHILP
DSSSLKTDLELKYKQTADFSARGFMPSTTAYPFDLPNAGTHNENFIF
GQSYYKASDGALFPLEVTVMLNKRLPDSRTSYVMTFLWSLNAGLAPE
TTQATLITSPFTFSYIREDDGGGSGGGSGGGSC;
c.
(SEQ ID NO: 8)
RGSHHHHHHGSKNSIALKNNTLWTGPKPEANSIIEYGKQNPDSKLTL
ILVKNGGIVNGYVTLMGASDYVNTLFKNKNVSINVELYFDATGHILP
DSSSLKTDLELKYKQTADFSARGFMPSTTAYPFDLPNAGTHNENFIF
GQSYYKASDGALFPLEVTVMLNKRLPDSRTSYVMTFLWSLSAGLAPE
TTQATLITSPFTFSYIREDDGGGSGGGSGGGSC;
d.
(SEQ ID NO: 9)
GSKNSIALKNNTLWTGPKPEANSIIEYGKQNPDSKLTLILVKNGGI
VNGYVTLMGASDYVNTLFKNKNVSINVELYFDATGHILPDSSSLKT
DLELKYKQTADFSARGFMPSTTAYPFDLPNAGTHNENYIFGQSYYK
ASDGALFPLEVTVMLNKRLPDSRTSYVMTFLWSLSAGLAPETTQAT
LITSPFTFSYIREDDGGGSGGGSGGGSC;
e.
(SEQ ID NO: 10)
RGSKNSIALKNNTLWTGPKPEANSIIEYGKQNPDSKLTLILVKNGG
IVNGYVTLMGASDYVNTLFKNKNVSINVELYFDATGHILPDSSSLK
TDLELKYKQTADFSARGFMPSTTAYPFDLPNAGTHNENFIFGQSYY
KASDGALFPLEVTVMLNKRLPDSRTSYVMTFLWSLNAGLAPETTQA
TLITSPFTFSYIREDDGGGSGGGSGGGSC;
or
f.
(SEQ ID NO: 11)
RGSKNSIALKNNTLWTGPKPEANSIIEYGKQNPDSKLTULVKNGGI
VNGYVTLMGASDYVNTLFKNKNVSINVELYFDATGHILPDSSSLKT
DLELKYKQTADFSARGFMPSTTAYPFDLPNAGTHNENFIFGQSYYK
ASDGALFPLEVTVMLNKRLPDSRTSYVMTFLWSLSAGLAPETTQAT
LITSPFTFSYIREDDGGGSGGGSGGGSC.

11) The composition of any one of embodiments 1-10 wherein the multimerization domain conjugates two polypeptides to form a homodimer.
12) The composition of any one of embodiments 1-10, further comprising one or more compounds conjugated to the composition.
13) The composition of embodiment 12, wherein the one or more compounds are selected from the group consisting of therapeutics, diagnostics and imaging agents.
14) The composition of embodiment 13, wherein the one or more compounds comprise at least one therapeutic, wherein the therapeutic is selected from the group consisting of antibodies, immunoconjugates, CAR T-cells, nanoparticles, chemotherapeutics, radioactive particles, viruses, vaccines, cellular immunotherapy therapeutics, gene therapy constructs, nucleic acid therapeutics and combinations thereof.
15) An isolated nucleic acid encoding the composition of any one of embodiments 1-10.
16) A recombinant expression vector comprising the isolated nucleic acid of embodiment 15.
17) A host cell comprising the recombinant expression vector of embodiment 16.
18) A pharmaceutical composition, comprising a composition according to any of embodiments 1-10; and a pharmaceutically acceptable carrier.
19) A method for enhancing therapeutic treatment, or diagnosis of a disorder associated with epithelial tissue, and/or imaging epithelial tissues, comprising administering to a subject in need thereof:
   a. a therapeutic for treatment of the disorder, a diagnostic, or an imaging agent; and
   b. the pharmaceutical composition of embodiment 18, in an amount sufficient to enhance efficacy of the therapeutic, diagnostic, and imaging agent.
20) The method of embodiment 19, wherein the disorder associated with human tissue is selected from the group consisting of solid tumors, irritable bowel syndrome, inflammatory bowel disorder, Crohn's disease, ulcerative colitis, constipation, gatroesophageal reflux disease, Barrett's esophagus, chronic obstructive pulmonary disease, asthma, bronchitis, pulmonary emphysema, cystic fibrosis, interstitial lung disease, pneumonia, primary pulmonary hypertension, pulmonary embolism, pulmonary sarcoidosis, tuberculosis, pancreatitis, pancreatic duct disorders, bile duct obstruction, cholecystitis, choledocholithiasis, brain disorders, psoriasis, dermatitis, glomerulonephritis, hepatitis, diabetes, thyroid disorders, cellulitis, infection, pyelonephritis, multiple sclerosis, transplant rejection and gallstones.
21) The method of embodiment 19, wherein the disorder associated with epithelial tissue is a solid tumor.
22) The method of embodiment 21 wherein the solid tumor is selected from the group consisting of breast tumors, lung tumors, colon tumors, rectal tumors, stomach tumors, prostate tumors, ovarian tumors, uterine tumors, skin tumors, endocrine tumors, cervical tumors, kidney tumors, melanomas, pancreatic tumors, liver tumors, brain tumors, head and neck tumors, nasopharyngeal tumors, gastric tumors, squamous cell carcinomas, adenocarcinomas, bladder tumors and esophageal tumors.
23) The method of any one of embodiments 19-22 wherein one or more compounds comprises at least one therapeutic, wherein the therapeutic is selected from the group consisting of antibodies, immunoconjugates, viruses, nanoparticles, chemotherapeutics, radioactive particle, vaccines, cellular immunotherapy therapeutics, gene therapy constructs, nucleic acid therapeutics and combinations thereof.
24) The method of any one of embodiments 19-22, wherein the therapeutic comprises a chemotherapeutic or a monoclonal antibody.
25) The method of any one of embodiments 19-22, wherein the therapeutic comprises an anti-tumor monoclonal antibody.
26) The method of embodiment 25, wherein the anti-tumor monoclonal antibody comprises an antibody selected from the group consisting of trastuzumab, cetuximab, pertuzumab, apomab, conatumumab, lexatumumab, bevacizumab, bevacizumab, denosumab, zanolimumab, lintuzumab, edrecolomab, rituximab, ticilimumab, tositumomab, alemtuzumab, epratuzumab, mitumomab, gemtuzumab ozogamicin, oregovomab, pemtumomab daclizumab, panitumumab, catumaxomab, ofatumumab and ibritumomab.
27) The method of any one of embodiments 19-26, wherein the disorder associated with epithelial tissue comprises a Her-2 positive tumor.
28) A method for improving delivery of a compound to an epithelial tissue, comprising contacting the epithelial tissue with
   a. one or more compounds to be delivered to the epithelial tissue; and
   b. a conjugated composition of any one of embodiments 1-10, or functional equivalent thereof, or the pharmaceutical composition of embodiment 18, sufficient to direct delivery of the one or more compounds to the epithelial tissue.
29) The method of embodiment 28 wherein the one or more compounds comprises a diagnostic or an imaging agent.
30) The method of embodiment 28 or 29, wherein the epithelial tissue comprises a solid tumor.
31) The method of embodiment 30, wherein the solid tumor is selected from the group consisting of breast tumors, lung tumors, colon tumors, rectal tumors, stomach tumors, prostate tumors, ovarian tumors, uterine tumors, skin tumors, endocrine tumors, cervical tumors, kidney tumors, melanomas, pancreatic tumors, liver tumors, brain tumors, head and neck tumors, nasopharyngeal tumors, gastric tumors, squamous cell carcinomas, adenocarcinomas, bladder tumors, and esophageal tumors.
32) A method for improving delivery of a substance to a tissue expressing desmoglein 2 (DSG2), comprising contacting the tissue expressing DSG2 with
   a. one or more compound to be delivered to the tissue; and
   b. linked to an amount of the recombinant protein of any one of embodiments 1-10, or functional equivalent thereof, or the pharmaceutical composition of embodiment 18, in an amount sufficient to enhance delivery of the one or more compounds to the tissue.

The Examples and preparations provided below further illustrate and exemplify the polypeptides of the present disclosure and methods of preparing such polypeptides and methods of conjugating such polypeptides, and uses thereof. It is to be understood that the scope of the present invention is not limited in any way by the scope of the following Examples and preparations.

### EXAMPLES

### Example 1

### General Techniques

Cloning and Production of JO-1 Derivatives.

All genes used for this study were designed in silico using the software Serial Cloner v2.4.1. First generation JO derivatives, designated GB1-10, were based on the original JO-1 sequence as described previously (Beyer, I. et al. Clin Cancer Res 18, 3340-3351, doi:10.1158/1078-0432.CCR-11-3213 (2012); Beyer, I. et al. Cancer Res 71, 7080-7090, doi:10.1158/0008-5472.CAN-11-2009 (2011)). The plasmid pET29a (Novagen) was linearized for Gibson cloning using outward facing primers pET29a_GB_UB (5′-TCCTCTCATATGTATATCTCCTTC) (SEQ ID NO: 15) and pET29_GB_DT (5′-CTTAATTAGCTGAAATCACTAGT) (SEQ ID NO: 16) using a 1/10000 dilution of plasmid as template in the PCR reaction. GB sequences were appended with nucleotides corresponding to the overlap regions of pET29a entry vector and were synthesized by Integrated DNA Technologies (Coralville, Iowa). Gibson cloning of gBlocks into the pET29a entry vector was using the Gibson Cloning Master Mix (New England Biolabs, Ipswich, Mass.) per manufacturer's instructions. Cloned GBs were sequence confirmed (Genewiz, South Plainfield, N.J.) before being transformed into E. coli Rosetta BL21 (DE3) (EMD Millipore, Darmstadt, Germany).

DSG2 Binding Western and DSG2 Shedding Assay.

The media for culturing cells was made by adding 50 mL of Fetal Bovine Serum (Hyclone, Lot # AYK176955), 5 mL of Pen Strep (Gibco, Ref #15140), and 5 mL of L-Glutamine (Gibco, Ref #35030) to 500 mL of RPMI Medium 1640 (Gibco, Ref #11875-093). HT29 cells were cultured overnight in a 12-well dish (10\hat{ }6 cells/well) in 2 mL of the RPMI medium. Each protein was then added to three wells with a final concentration of 5 μg/mL. No protein was added to the three control wells. Two hours later, cells were rinsed with DPBS/Modified (Hyclone, Lot # AAE202584) and 2 mL of new media were added. At each time point (2 hr, 4 hr, 12 hr, 24 hr, 48 hr), 110 μl aliquots of media were collected from each well and replaced with 110 μl of new media. The samples were frozen at −20° C. until an ELISA was run using anti-Human Desmoglein-2 Antibody (R&D System, Catalog # AF947) or a murine anti-human Desmoglein-2 monoclonal Antibody 6D8 (AbD Serotec, Cat # MCA2272). The graph reflects an average of the three wells for each protein.

Viral inhibition assay. HeLa cell suspensions were confirmed to be >98% viable, their concentration adjusted to 2×105 cells/ml, and then plated in 96 well plates with 200 μL per well (Corning, Inc., Corning, N.Y.). Following 18 h incubation at 37° C., 5% CO2, 62.5 μL of protein diluted in complete DMEM were added to each well in quadruplicate. A total of 11 half log dilutions were tested in quadruplicate for each protein. Following 1 hr incubation to allow proteins to bind the HeLa cells, 50 μL of Ad3 virus expressing GFP were added in complete DMEM at an MOI of 100 and incubated for 2 hours to facilitate viral entry. After 2 hours, media was removed, cells washed with DMEM, and the plates incubated at 37° C. and 5% CO2 for 16-18 hours. On the next day, plates were read with a bottom read orientation at 475 nm Excitation and 505 nm Emission using a SpectraMax i3x plate reader (Molecular Devices, Inc., Sunnyvale, Calif.) utilizing Softmax Pro software. Data were plotted using Graphpad Prizm (GraphPad Software, Inc., La Jolla, Calif.) and the IC50 determined using a 4 parameter non-linear fit of the sigmoidal curves.

SPR analysis of binding affinities to DSG2. SPR analysis was performed at 25° C. using a Biacore 3000 instrument (GE Healthcare, Pittsburgh, Pa.). JO4 and GB7 were diluted to 10 μg/mL in 10 mM sodium acetate pH 4.5 and immobilized on a CM5 sensor chip (approximately 6.000 RU) by standard amine coupling chemistry. hDSG2 binding was measured in 10 mM HEPES, 150 mM NaCl, 0.005% surfactant P20, 2 mM CaCl₂) pH 7.4 at a flow rate of 15 μL/min. A total of 5 dilutions of hDSG2 ranging from 17.4 nM down to 0.87 nM were tested in triplicate. hDSG2 (10 μg/mL) was captured on a CM5 sensor chip using the amine coupling chemistry until a coupling level of 6.000 RU was obtained. GB7 and GB3 binding were measured in 10 mM HEPES, 150 mM NaCl, 0.005% surfactant P20, 2 mM CaCl₂) pH 7.4 at a flow rate of 15 μl/min. A total of 3 dilutions of GB7 ranging from 19 nM down to 9.5 nM and 4 dilutions of GB3 ranging from 8.3 nM down to 0.83 nM were tested. The surfaces were regenerated by pulse injection of 5 mM EDTA. The signal recorded on reference flow cell without protein were subtracted from that obtained on protein. For association data, proteins were injected at a flow rate of 15 μL/min for 180 seconds. Following the association phase, buffer was flowed at 15 μL/min for an additional 150 seconds to measure dissociation. Binding curves were analyzed using BIAEvaluation software (GE Healthcare) and data was fit to a 1:1 Langmuir interaction model. Affinities were mathematically derived by subtracting the dissociation rate by the association rate.

Transmission Electron Microscopy.

Negative-stain electron microscopy. Recombinant GB proteins were visualized by negative-stain electron microscopy. The standard mica-carbon preparation was used with protein at 0.1 mg/ml. Samples were stained using 2% (wt/vol) uranyl acetate and visualized on a JEOL (JEM-1200EXII) electron microscope at 100 kV. Images were acquired and analysed by the digital micrograph software (Gatan).

Size Exclusion Chromatography and Multi-Angle Light Scattering Analysis (SEC-MALS).

Size-exclusion chromatography was performed on an Agilent 1200 HPLC system (Agilent Technologies, Santa Clara, Calif.) with the GE HealthCare Superdex 200 Increase 10/300 GL prepacked column and an isocratic 1×TBS gradient at 1 mL/min. JO4, GB3, and GB7 were at concentrations of 2.5 mg/mL, 2.8 mg/mL, and 2.5 mg/mL in PBS with 5% glycerol, respectively, with 100 □L of each injected. Protein was measured via UV absorbance at 280 nm and sizes determined based on multi-angle light scattering (MALS) analysis was performed using a miniDAWN TREOS and Optilab T-rEX apparatus with ASTRA software (Wyatt Technologies, Santa Barbara, Calif.). For reduction of proteins, DTT was added to an initial concentration of 1.6 mM, proteins were incubated at 50° C. for 10 min, and then an additional 1.6 mM DTT was added to maintain the reduced conformations until the SEC-MALS analysis could be performed. Relative mass concentration was calculated by importing chromatograms into Graph 4.3, generating a 5-period moving average function, and measuring peak areas. Figures were generated in GraphPad Prism 6.

Conjugation of JOC-x to PEG-Biotin or DOTA and Labelling with Europium.

JOC-x was conjugated to both PEG2-biotin and PEG₁₁-biotin using the EZLink™ Maleimide-PEG2-Biotin and EZLink™ Maleimide-PEG11-Biotin kits respectively (ThermoFisher Scientific, Waltham, Mass., USA). JOC-x protein was first reduced by adding dithiothreitol (DTT) at a concentration of 10 mM and incubated at 37° C. for 1 hour. DTT was then removed by running the solution through Zeba desalting columns (ThermoFisher Scientific, Waltham, Mass., USA). The maleimide-PEG_{2/11}-Biotin was added to JOC-x at a molar ratio of 20:1 (biotin:JOC-x) and allowed to react, rotating for 24 hours at room temperature. Excess PEG2/11-Biotin was then removed by Zeba desalting columns. Biotin conjugation was confirmed by SDS-PAGE analysis. LanthaScreen™ Eu-Streptavidin (ThermoFisher Scientific, Waltham, Mass., USA) was used as the streptavidin-europium (SA-Eu) payload. JOC-Biotin and SA-Eu were mixed at a molar ratio of 1:1 in solution, allowed to bind at room temperature for 30 minutes, and then subsequently run on a SDS-PAGE gel. The gel was then transferred to a PVDF membrane and imaged using the SpectraMax i3x imager (Molecular Devices, San Jose, Calif., USA), at an emission wavelength of 616 nm.

Conjugation of DOTA to JOC-x.

In preparation for conjugation of DOTA to JOC-x, JOC-x was reduced by adding DTT at a concentration of 0.1 μg/mL and incubating at 37° C. for 1 hour. DTT was then removed by running the solution through Zeba desalting columns. Maleimido-mono-amide-DOTA (Macrocyclics, Dallas, Tex., USA) was added to reduced JOC-x at a molar ratio of 10:1 (DOTA:JOC-x) and allowed to react at room temperature overnight. DOTA conjugation to JOC-x was confirmed by SDS-PAGE and mass spectrometry. Europium ions were sourced from EuCl₂.6H₂O (Sigma-Aldrich, St. Louis, Mo., USA). The JOC-DOTA was buffer exchanged into an ammonium acetate buffer at pH 5.8 to promote chelation of Eu. EuCl₂.6H₂O was then added at a molar ratio of 10:1 (EuCl₂.6H₂O:JOC-DOTA) and allowed to react at 37° C. for 1 hour and left at room temperature for 24 hours. A 10× molar excess (of Eu added) of ethylenediaminetetraacetic acid (EDTA) was then added to chelate unreacted Eu. Running the solution through a Zeba desalting column (ThermoFisher Scientific, Waltham, Mass., USA) eliminated residual europium and restored the buffer to a phosphate pH 7.0 buffer. Eu chelation was confirmed by resolving the proteins on SDS-PAGE, transferring to PVDF membrane, and imaging phosphorescence using a SpectraMax i3X (Molecular Devices, San Jose, Calif., USA) at an emission wavelength of 616 nm.

Conjugation of JOC-x to poly(I:C) and measurement of TLR3 signaling. Low molecular weight (LMW) poly(I:C) (Invivogen) was resuspended to a final concentration of 10 mg/mL in 10 mM sodium phosphate, 150 mM NaCl, 10 mM EDTA, pH 7.2. Next, 0.75 mL of LMW poly(I:C) was mixed with 125 mg of 1-ethyl-3-(3-dimethylaminopropyl)carbodiimide (EDC) in a 15-mL Falcon tube. Finally, 0.5 mL of 250 mM ethylenediamine in 100 mM imidazole was added to the poly(I:C), thoroughly vortexed, and 2 mL of 100 mM imidazole was added and allowed to react overnight at RT. The following day, unreacted EDC and ethylenediamine were removed from the poly(I:C)-NH2 by two passes through 5 mL 7k MWCO Zeba™ Spin Desalting Columns (Thermo Scientific) that were equilibrated with 100 mM sodium phosphate, 150 mM NaCl, 5 mM EDTA, pH 7.2. Purified poly(I:C)-NH2 was then incubated with a 100-fold molar excess of sulfosuccinimidyl 4-(N-maleimidophenyl)butyrate (sulfo-SMPB) (Thermo Scientific) for 1 hour at RT before removing excess sulfo-SMPB with two passes through Zeba™ columns as described above. Simultaneously, JOC-x was incubated with 5 mM tris(2-carboxyethyl)phosphine (TCEP) for 30 minutes at RT before removing excess TCEP with two passes through Zeba™ columns. Finally, poly(I:C)-NH2 was incubated with JOC-x at a 1:2 molar ratio at RT overnight. Conjugation was confirmed by nucleic acid gel electrophoresis, SDS-PAGE, and Western blot. TLR3 signaling was measured using a commercial HEK-Blue™ hTLR3 Assay (Invivogen, San Diego, Calif.) according to manufacturer's specifications. TLR3 activation resulted in NFκB- and AP1-induced production of the reporter gene Secreted Embryonic Alkaline Phosphatase (SEAP).

### Example 2

### Generating a Protein without Internal Cysteines and with a Free Cysteine for Conjugation

In this example, JO-1 is modified to remove the cysteine residues and a free cysteine for conjugation is added.

The amino acid sequence of the parent molecule JO-1 and key residues known to enhance binding are shown in FIG. 1a. The 6×-HIS tag, the dimerization domain, and the Ad3 Knob region are shaded in grey, blue, and yellow respectively in FIG. 1a. JO-1 has two native cysteine residues (amino acids 80 and 255) which, based on the crystal structure, are spatially distant and unlikely to form intramolecular disulfide bridges. Removal of the internal cysteines would be a means to limit oxidation and covalent aggregation while allowing a designer sulfhydryl-based site to be added later.

A total of 10 genes were constructed based on JO-1, termed GB1-10, and are shown in Table 1.

TABLE 1
Phenotypic and schematic representation
of JO-derived protein constructs

| Protein | DSG-2 binding | VIA IC₅₀ (μg/mL) |
|---|---|---|
| JO-4 | Yes | 0.067 |
| GB1 | Yes | 0.025 |
| GB2 | Yes | 0.030 |
| GB3 | Yes | 0.029 |
| GB4 | Yes | 0.037 |
| GB5 | Minimal | 0.241 |
| GB6 | n/a | n/a |
| GB7/JOC-x | Yes | 0.032 |
| GB8 | Yes | 0.012 |
| GB9 | No | >10 |
| GB10 | Yes | 0.026 |

Following codon optimization of JO-1 (GB1) to maximize expression in E. coli, GB2 (JO-1) was constructed by mutating the native internal cysteines to serines. Next, a single cysteinyl residue was introduced at various positions throughout the protein to facilitate directed conjugation without disruption of active quaternary conformations (i.e. trimer-dimers). For two of the proteins, a cysteinyl residue was added at the C-terminal position following a flexible glycine serine linker (G₄S)₃. The single cysteine was also added to the N-terminus either before or immediately following the His tag resulting in proteins GB4 and GB10, respectively. To confirm an earlier finding suggesting that the H-I loop (PECTT, underlined in FIG. 1Aa) is required for stabilization of DSG2 binding, a cysteine was introduced within this loop resulting in GB6. The locations of insertion of all cysteinyl residues within JO-1 GBs are indicated in FIG. 1Aa by black arrows.

### Example 3

### The Free Cysteine Functions as a Covalent Mediator of Multimerization

In this example, function of the free cysteine in JO-1 derivatives without the dimerization domain is examined.

It is unclear what role the dimerization domain (DD), which does not exist in the native viral knob protein, plays in formation of trimers, trimer dimers, higher order multimers, or aggregates. The DD contains 5 repeats of seven amino acids, and each repeat contains two lysines. These lysine-heavy regions could potentially result in ribosomal stalling during translation as the cell becomes depleted of tRNAs encoding this amino acid. Additionally, the DD has a predicted isoelectric point (pI) of ˜10.91, making it very basic in nature and taking on a strong positive charge at neutral pHs. Addition of the DD to the Ad3 knob to generate JO-1 resulted in a high predicted pI of ˜8.65, whereas the pI of the native knob protein is ˜7.32. The high pI and repetitive nature of the DD suggested that removal of this domain would likely 1) lower aggregation beyond the levels observed with the Cys-deleted GBs, 2) enhance protein expression levels, and 3) reduce the protein pI significantly leading to lower endotoxin binding. To test this hypothesis, GB5, GB7, GB8 and GB9 were all constructed by deleting the DD. GB5 (ΔDD) contained no cysteines at all, GB7 (ADD) was constructed similar to GB3 with the C-terminal (G4S)3 followed by a terminal cysteine residue, GB8 (ADD) contained a cysteine immediately following the His tag, and GB9 (ADD) was similar to GB6 with a cysteine in the H-I loop.

Eliminating the dimerization domain confers multiple production advantages. A total of 10 second generation JO-1 derivatives, described in Table 1 and designated GB1-10 were generated as gBlocks (Integrated DNA Technologies (Coralville, Iowa)), four of which contained no DD. GB3 and GB7 were generated as JO-4 versions by introducing the V239D mutation. All 12 GB proteins were successfully produced in E. coli with the exception of GB6. For GB6, 10 sequence verified clones failed to express protein, and this clone was eliminated from further analysis. Expression levels of GBs containing the DD were similar to that of JO-1 and ranged from 0.5-5 mg/L. Attempts to optimize codons throughout the DD did not increase expression relative to JO-1 with the original DD sequence. As predicted, the expression levels of all clones not containing the DD were significantly higher than JO-1 and ranged from ˜5-20 mg/L.

Initial endotoxin levels following purification of all GBs containing the DD were high, ranging from 80,000 to ˜1,500,000 EU/mg, with an average of 691,300 EU/mg as determined by the Limulus Amoebocyte Lysate (LAL) method (Charles River Laboratories, Inc., Charleston, S.C.). By contrast, endotoxin levels of DD-deleted clones were 100 to 1000 fold lower with endotoxin values of multiple production runs ranging from ˜3000-20000 EU/mg. Most significantly, multiple production runs of GB7 resulted in an average endotoxin level of only 2500 EU/mg which, as described below, was reduced further following final purification and polishing steps. Clones containing a DD bound endotoxin much more tightly. For example, with clones containing a DD, multiple attempts using a variety of endotoxin removal kits and resins failed to remove endotoxin to below 5000 EU/mg, suggesting a nearly irreversible binding. A protocol utilizing multiple ion exchange and hydrophobic interaction chromatography steps successfully reduced endotoxin to below 10 EU/mg, but was accompanied by significant protein loss. This suggests that the interaction is so tight that the majority of endotoxin removed by the chromatography resins likely is still bound to the protein. As for GBs not containing the DD, such as GB7, a single pass through Pierce™ High Capacity Endotoxin Removal Spin Column (Life Technologies Corp., Grand Island, N.Y.) removed bound endotoxin to levels less than 10 EU/mg. Moreover, the endotoxin was removed with minimal protein loss (data not shown) suggesting weaker binding between GB7 and endotoxin.

Functional Responses are Preserved.

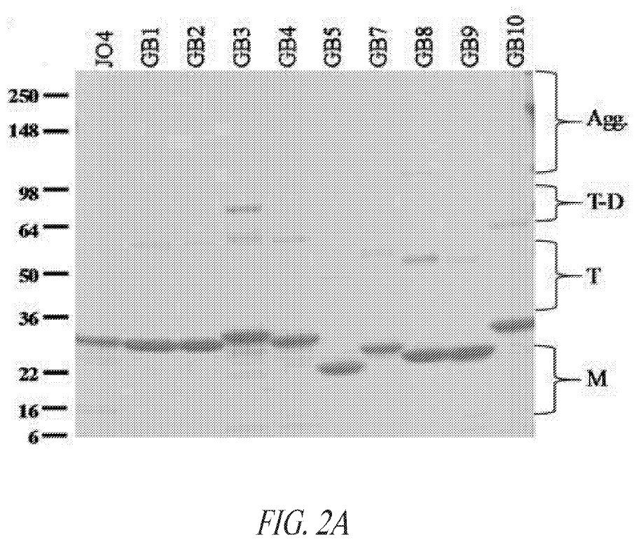

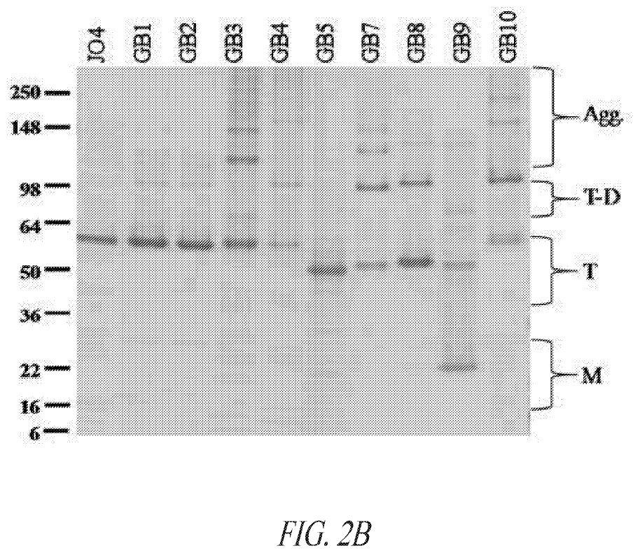

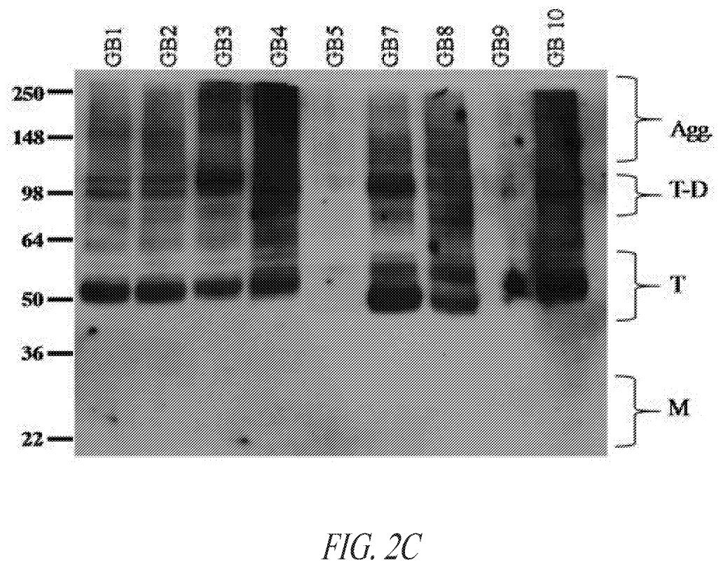

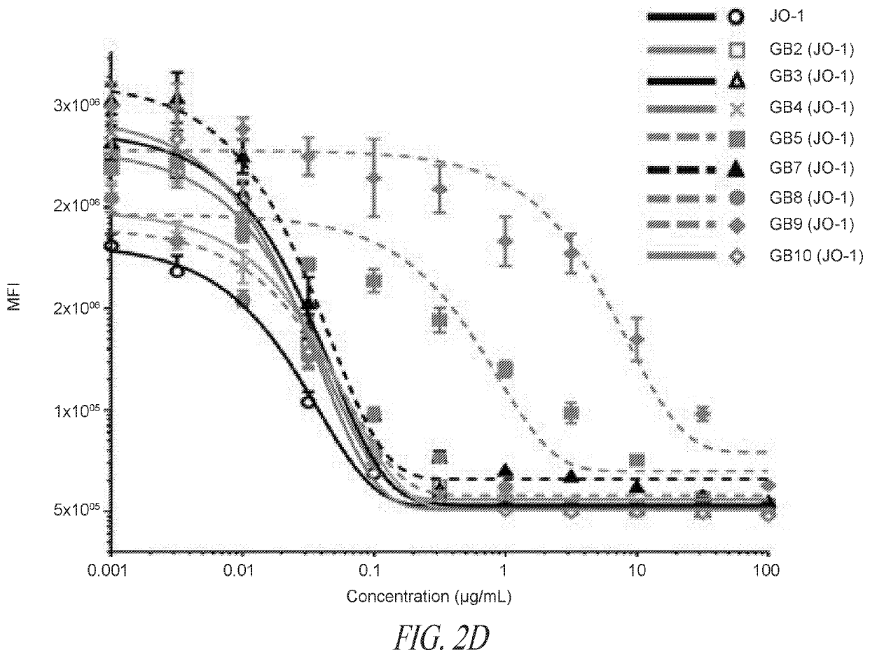

The remaining GBs yielded protein of the expected molecular weight on boiled and reduced SDS-PAGE as shown in FIG. 2a. Proteins were also analyzed by non-reduced and un-boiled SDS-PAGE (FIG. 2b). While not true native gels, this analysis allows this family of proteins to retain multimeric forms (e.g. trimers, trimer-dimers, multimers, and aggregates) and is an indicator of the likelihood of activity. Previous data have shown inactivated JO-1 will not form higher-order multimers on non-reduced SDS-PAGE, and this protein will neither bind DSG2 nor inhibit viral entry. Non-reduced JO-1 protein will typically form multiple bands on SDS-PAGE gels including a trimer (T) at ˜60 kDa, a trimer-dimer (T-D) at ˜100 kDa, and multimers (multi.) or aggregates (Agg.) at >150 kDa with almost no monomeric form being visible on the gel at 27.9 kDa. Reduced JO-1 will form a single band at 27.9 kDa on SDS-PAGE. All GB proteins formed trimers, trimer-dimers, and aggregates with the exception of GB5 and GB9. GB5 appeared to form a higher MW band in the trimer region, but did not to produce higher MW isoforms such as trimer-dimers. GB9 appeared to produce the higher MW isoforms, although a significant amount of monomer was consistently observed in the non-reduced state.

To confirm the presence of active conformations, the panel was tested for binding to human DSG2 protein by Western blot as shown in FIG. 2c. This assay has been used previously to assess protein function given that only the trimeric and larger forms of the protein will bind native DSG2. A prominent band was visible at the expected molecular weights for all JO-1 derivatives except GB5 and GB9. This result was not unexpected for GB5 given the lack of higher MW multimers beyond the trimer. GB9, despite forming multiple higher order conformations, failed to bind DSG2 confirming earlier reports on the importance of the H-I loop in this interaction. One especially interesting observation was the binding of GB7 and GB8 to DSG2 given that these proteins only differ from GB5 by the presence of a single cysteine linker domain at the C- or N-termini, respectively.

Next the ability of the GBs to bind to DSG2 in vitro in HeLa cells and to block entry of fluorescent Ad3 virus using the viral inhibition assay (VIA) [Wang, H. et al. J Virol 87, 11346-11362, doi:10.1128/JVI.01825-13 (2013)] was assessed. This assay is a robust predictor of in vivo activity. For this study, the typical flow cytometric VIA assay was adapted to 96-well microplates allowing for much higher sensitivity at very low protein concentrations. The resulting sigmoidal inhibition curves can then be fitted to a four parameter non-linear regression analysis model resulting in the IC50 values which represent the concentration, in μg/mL, at which 50% of viral inhibition is observed. FIG. 2d shows the Ad3 viral inhibition curves for each of the 9 JO-1 derived GB proteins and the JO-1 parental control. Seven of the 9 GBs had binding curves similar to the JO-1 control, with GB5 showing significantly lower and GB9 showing almost no Ad3 virus inhibition. The IC₅₀ values of the entire GB panel are reported in Table 1. Most GB proteins were within the standard deviation range of JO-1 with the exception of GB8, which resulted in approximately twofold greater viral inhibition relative to JO-1. GB5 and GB9 showed the least inhibition resulting in 5-fold and >100-fold lower inhibition, respectively.

Removal of the internal cysteines in GB2 did not affect, and in fact enhanced, activity of the protein as determined by DSG2 binding and viral inhibition. GB5, lacking both cysteines and the DD, failed to form higher MW isoforms and did not bind DSG2 in our Western blot analysis, yet still inhibited virus albeit at a much lower level. Similar to the DSG2 binding assay, both GB7 and GB8, which differ from GB5 by only one cysteine, inhibited viral entry at comparable levels relative to JO-1. We concluded that GB5 may be binding a very small amount of DSG2 allowing for the partial inhibition. As expected, perturbation of the H-I loop in GB9 resulted in no discernable DSG2 binding by Western blot and very little viral inhibition despite forming higher MW multimers.

### Example 4

### Down Selection of Next Generation Junction Openers

Based on production levels, DSG2 binding, and viral inhibition results, GB3 and GB7 were down selected for further characterization of DSG2 shedding and aggregation states. Affinity enhanced versions of GB3 and GB7 (SEQ ID Nos: 17-18) containing the V239D mutation found in JO-4 were produced and used for all future analyses. GB3 was chosen to assess the role of cysteines in formation of higher order multimers, while GB7 was chosen to assess the role of the DD in the same process. GB7 was prioritized over GB8, despite GB8 have a lower IC50, due to the location of the single cysteinyl residue. Specifically, having a cysteine at the C-terminus would be advantageous for conjugation of the protein to effector molecules (drugs, antibodies, or isotopes) as well as delivery systems such as nanoparticles. With respect to GB8, conjugation to a cysteine proximal to the 6×-his tag could pose steric hindrances and could affect downstream purification efforts.

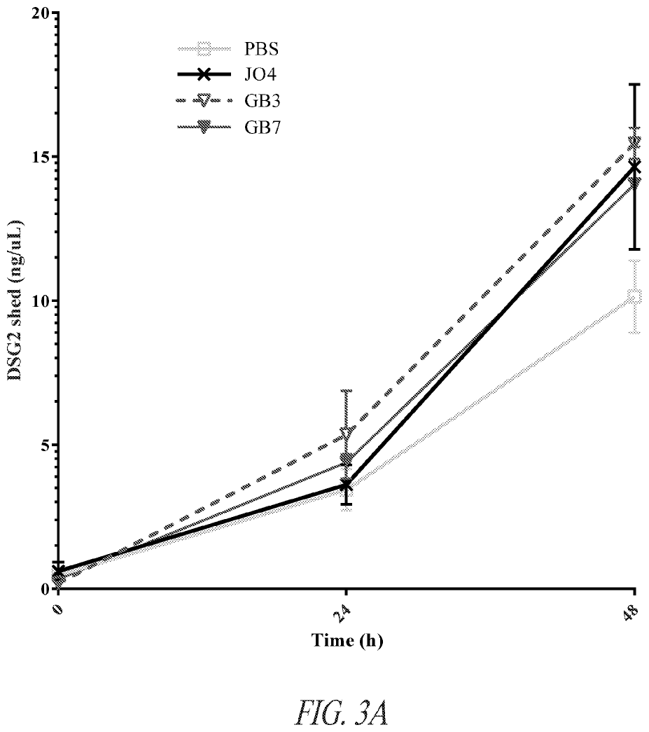

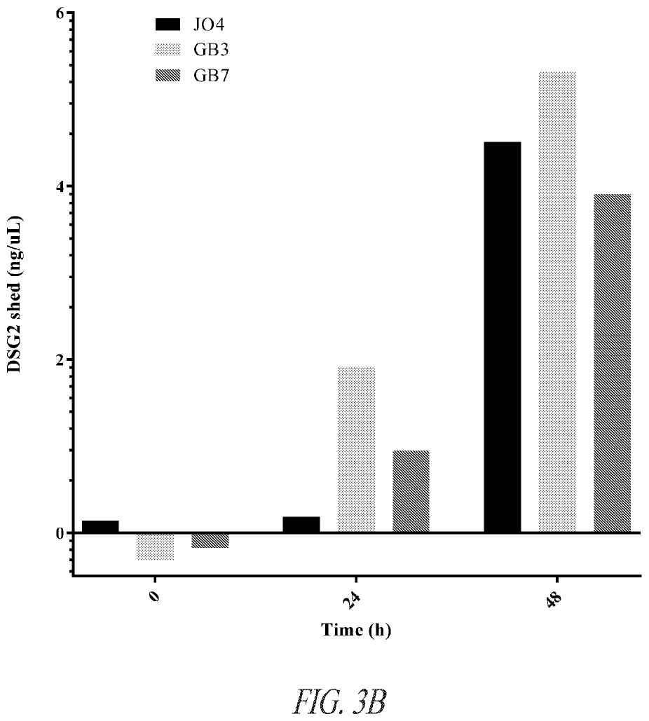

DSG2 is a membrane glycoprotein of epithelial cells and promotes formation of tight junctions between cells. Many epithelial cancers are known to highly upregulate the production of DSG2 resulting in formation of a network of cellular “staples” reminiscent of poorly organized junctions that make tumors difficult to permeate by cells and treatments. DSG2 is shed by the action of matrix metalloproteinases in a process enhanced by treatment with JO-1. During this junction opening process a significant amount of DSG2 is shed from the cell surface into the surrounding media, and the amount shed can be quantified using a simple ELISA. FIG. 3a shows the relative amounts of DSG2 shed in response to control (PBS) treatment as compared to treatment with the affinity enhanced JO-4, GB3, or GB7. Over the course of 48 hours, all three junction opener proteins resulted in increased DSG2 shedding. The amount of DSG2 shedding induced by control or junction-opener proteins is plotted in FIG. 3a. Because some amount of DSG2 shedding is natural, as observed with PBS treated cells, the levels of DSG2 shed following subtraction of the level observed in the PBS treated cells were plotted (FIG. 3b). From the blank subtracted junction openers, JO-4, GB3, and GB7 resulted in 1.44-, 1.52-, and 1.39-fold higher DSG2 shedding, respectively, relative to the PBS treated monolayers.

To compare the affinities and association/dissociation rates of JO derivatives to DSG2, SPR (surface plasmon resonance) analysis was performed by immobilizing recombinant human DSG2 onto a sensor chip and flowing the junction opener proteins over the surface. As shown in Table 2a, this analysis resulted in affinity measurements of 11.4 nM, 0.11 nM, and 0.58 nM for JO-4, GB3, and GB7, respectively.

TABLE 2a
Biacore analysis of binding to hDSG-2:
Rates following immobilization of hDSG2 protein

| Protein | Kₐ (M/S) | K_{d} (1/s) | K_{D} (nM) |
|---|---|---|---|
| JO4 | 2.32 × 10⁵ | 2.64 × 10⁻³ | 11.4 |
| GB3 | 4.4 × 10⁶ | 5.0 × 10⁻⁴ | 0.11 |
| GB7 | 1.4 × 10⁶ | 8.3 × 10⁻⁴ | 0.58 |

The high affinity of GB3 was due to an improvement of its association rate compared to JO-4 or GB7. The dissociation rates for the three proteins were similar (ranging from 4.6-7.5×10-4). However, in performing this assay, the JO-4 protein was found to bind to the sensor surface somewhat non-specifically, possibly due to aggregation, and was not easily regenerated from the sensor surface. By contrast, GB7 showed much lower nonspecific binding and was easily regenerated from the sensor surface. In order to investigate the impact of aggregation on binding rates, the SPR assay was repeated by immobilizing JO-4 and GB7 directly on the sensor chip and flowing rhDSG2 over the surface (GB3 was not tested in this format). In this format, the affinity of JO-4 to DSG2 showed a nearly 20-fold improvement from 11.4 nM to 0.58 nM while the affinity of GB7 for DSG2 was reduced only twofold from 0.58 nM to 1.19 nM (Table 2b). The 20-fold improvement in affinity by JO-4 was due to a 3.4-fold faster on-rate and a 5.7-fold more stable dissociation rate when the JO-4 was directly immobilized to the sensor surface and is likely due to impaired diffusion kinetics caused by large aggregates.

TABLE 2b
Biacore analysis of binding to hDSG-2:
Rates following immobilization of JO-derivative proteins

| Protein | Kₐ (M/s) | K_{d} (1/s) | K_{D} (nM) |
|---|---|---|---|
| JO4 | 7.9 × 10⁵ | 4.6 × 10⁻⁴ | 0.58 |
| GB7 | 6.3 × 10⁵ | 7.5 × 10⁻⁴ | 1.19 |

### Example 5

### Junction Openers without the Dimerization Domain Exhibit Minimal Aggregation

To investigate the presence of aggregates and various multimeric states, SEC-MALS analysis was performed. This method combines size exclusion chromatography (SEC) for separation of homogenous populations into distinct peaks with multi-angle light scattering (MALS) analysis to determine the both the MW and percentages of each species. Each protein was tested in the native (presumably oxidized) form or following reduction with 2 mM DTT to reduce disulfide bonds.

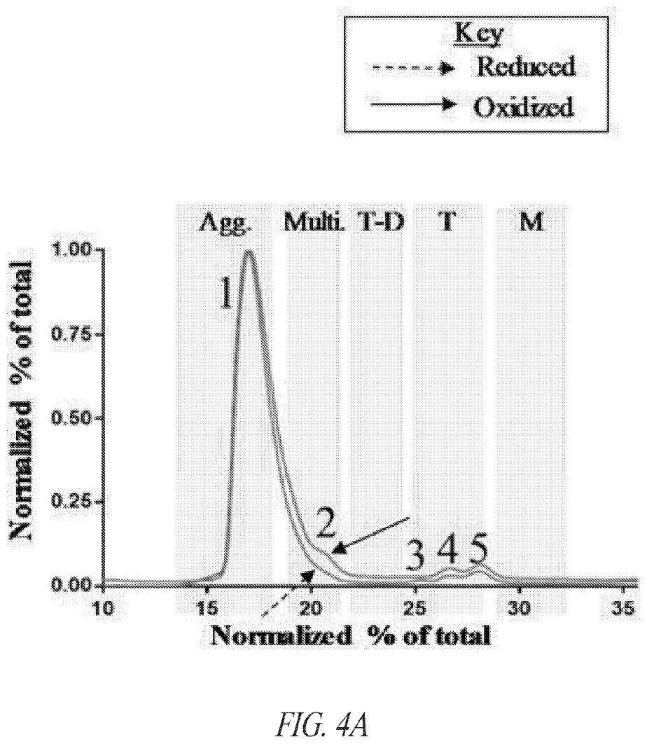

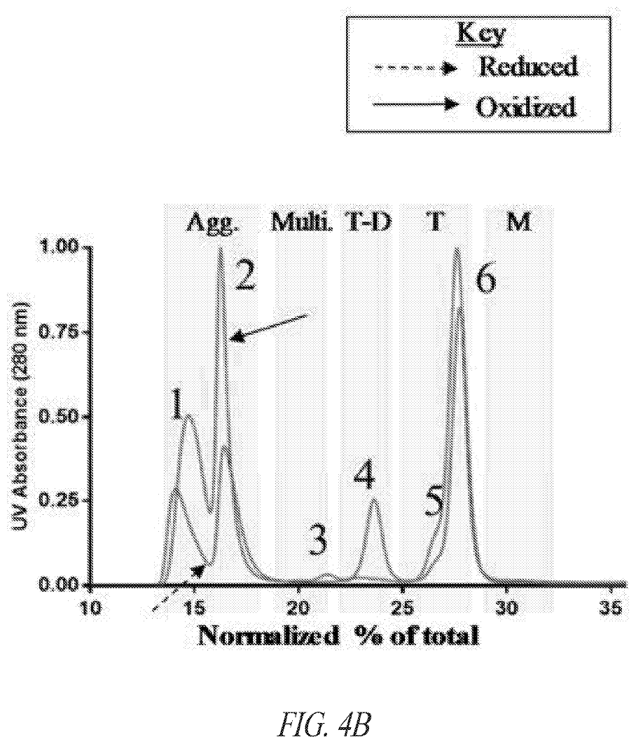

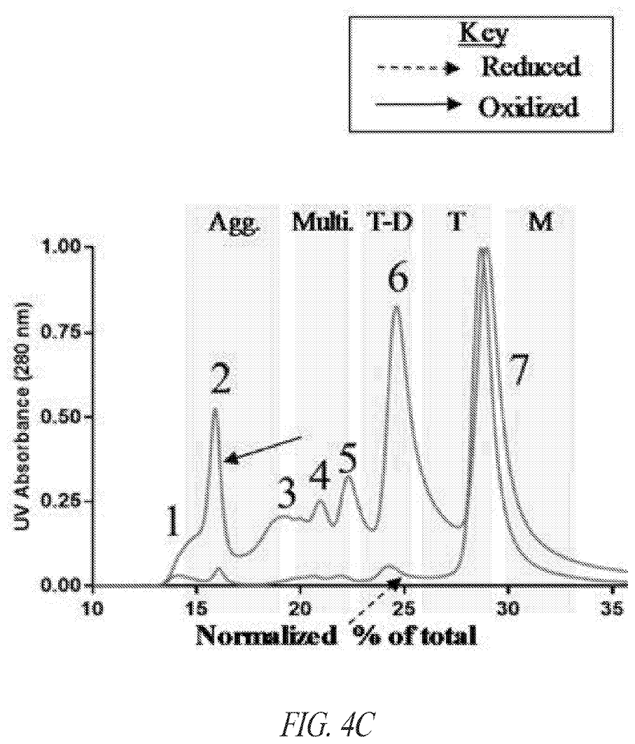

The results of the SEC-MALS analysis is shown in FIG. 4 whereby the shaded and numbered boxes indicate where the (1) aggregates, (2) multimers, (3) trimer dimers, and (4) trimers were found based on MALS size determination. MALS analysis was used to estimate average molecular weights of the proteins within each peak as well as the approximate percentages of each species. Results of the MALS analysis are summarized in Table 3 and include the predicted sizes of each species, the observed sizes, and the percentages of each species.

TABLE 3
Observed mass and percentages of protein states determined by MALS analysis

| | | | | | | Observed % of | |
|---|---|---|---|---|---|---|---|
| | Probable | | Predicted | Observed MW | | species | |
| Peak | Species | Protein | MW (kDa) | Oxidized | Reduced | Oxidized | Reduced |
| 1 | Aggregates | JO4 | >503 | 1120 | 1976 | 82 | 88 |
| | | GB3 | >516 | 610 | 23100 | 49 | 37 |
| | | GB7 | >434 | 3540 | 27600 | 17 | 2 |
| 2 | Multi- | JO4 | 251-503 | 487 | 727 | 15 | 9 |
| | mers | GB3 | 258-516 | 561 | 8950 | 2 | 1 |
| | | GB7 | 217-434 | 1360 | 1050 | 26 | 4 |
| 3 | Trimers- | JO4 | 167 | n.d.¹ | n.d.¹ | 2 | 2 |
| | Dimers | | | | | | |
| | | GB3 | 172 | 297 | n.d.¹ | 11 | 1 |
| | | GB7 | 145 | 222 | 298 | 22 | 3 |
| 4 | Trimers | JO4 | 84 | n.d.¹ | n.d.¹ | 2 | 1 |
| | | GB3 | 86 | 75 | 65 | 38 | 61 |
| | | GB7 | 72 | 67 | 69 | 36 | 91 |

n.d.¹: not determined due to insufficient light scattering signal intensity

As shown in FIG. 4A, JO-4 exhibited a dominant peak in the region corresponding to aggregates with approximately 97% of the protein found as aggregates or high MW multimers and only ˜4% existing as trimers or trimer dimers. Reduction of JO-4 with DTT did not significantly alter the aggregative phenotype. Removal of internal cysteines in GB3 (FIG. 4B) reduced the aggregation phenotype and increased the amounts of multimers and trimers. Specifically, GB3 had approximately 49% aggregates when oxidized and 37% when reduced, and the presence of trimers and trimer dimers increased to 49% in the oxidized state and 62% following reduction. Removal of the DD in GB7 (FIG. 4C) yielded the least amount of aggregates with only 17% or 2% aggregates in the oxidized and reduced states, respectively. Moreover, GB7 formed 58% trimers and trimer dimers in the oxidized state and when reduced yielded 91% trimers. Oxidized GB7 clearly showed the greatest amount of transitional states compared with either JO-4 or GB7 and was the only protein which could be effectively reduced nearly completely to the trimeric conformation. Interestingly, none of the 3 proteins could be found in their monomeric form even with DTT reduction similar to what is observed with non-reduced SDS-PAGE analysis which also resulted in almost no visible monomers.

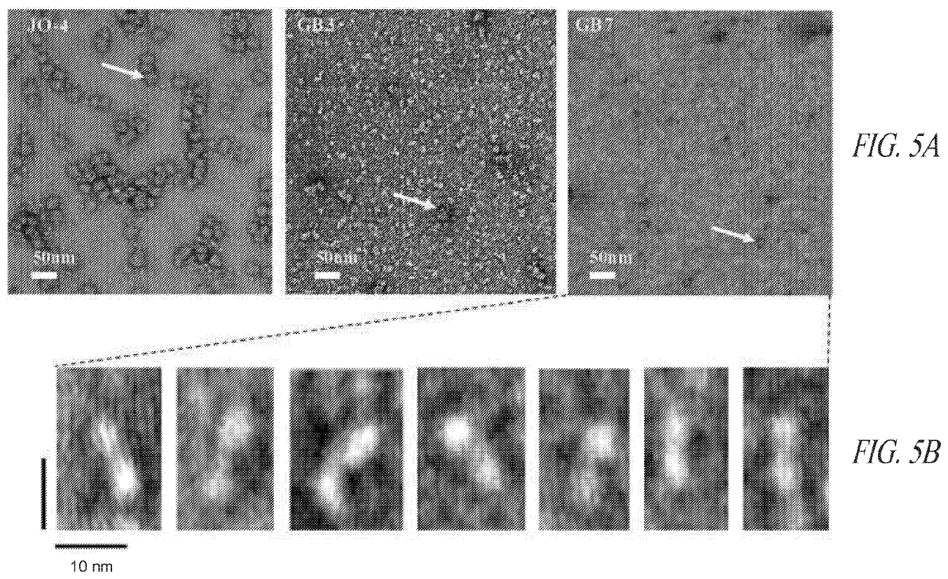

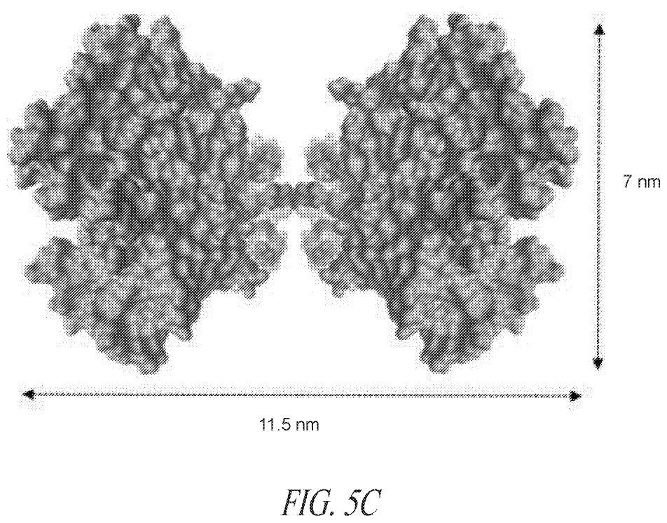

Further evidence of the aggregative phenotypes was confirmed by performing uranyl acetate negative staining of protein smears and imaging by transmission electron microscopy (TEM). As shown in FIG. 5A, JO-4 shows relatively uniform higher order structures approaching 50 nm in diameter (white arrows) and resembling penton dodecahedra but likely made of much more than 12 fibers. By comparison, GB3 exhibits far fewer high MW aggregates compared to JO-4. This phenotype is even more pronounced with GB7 as only a few aggregates are observable in this field. The white specks in the background were presumed to be trimers and/or trimer-dimers, the latter of which having been described as resembling barbell-like structures, and are prevalent throughout the images.

Fine resolution enhancement of seven of these white specks from the GB7 image (FIG. 5B) showed a structure significantly resembling barbell-shaped associated with trimer-dimers. The size and conformation of the trimer structures is similar to those found in the atomic structures described previously for Ad3 fiber knob based on crystallography data. Using the Visual Molecular Dynamics program (VMD, U. of Illinois Urbana-Champaign) and the known x-ray crystallography coordinates for JO-4, a model of the trimer-dimer structure is shown in FIG. 5C. VMD software estimated the size of the trimer-dimer to be approximately 7 nm wide and 11.5 nm long, which is consistent with the size observed in the TEM images shown in FIG. 5B. Specifically, they appear similar in size and shape to the proposed barbell structure modeled in FIG. 5C.

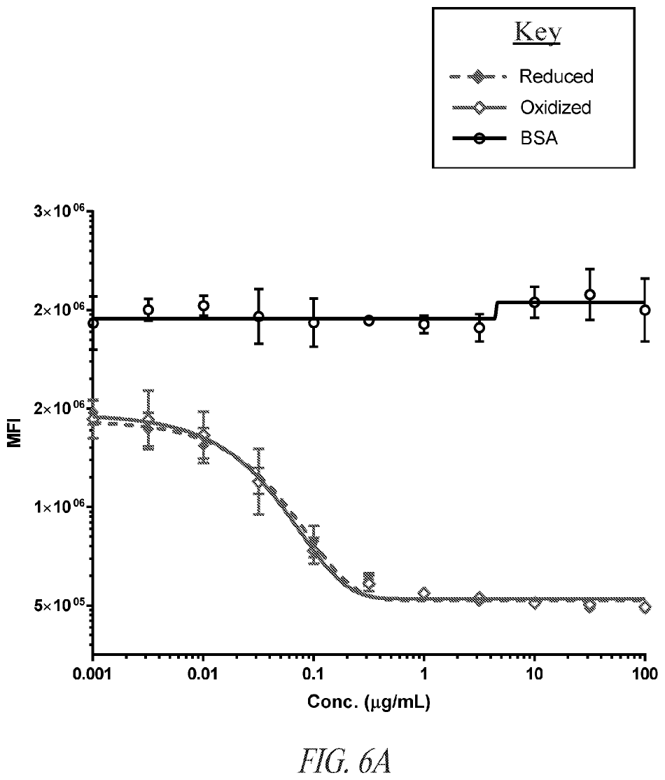

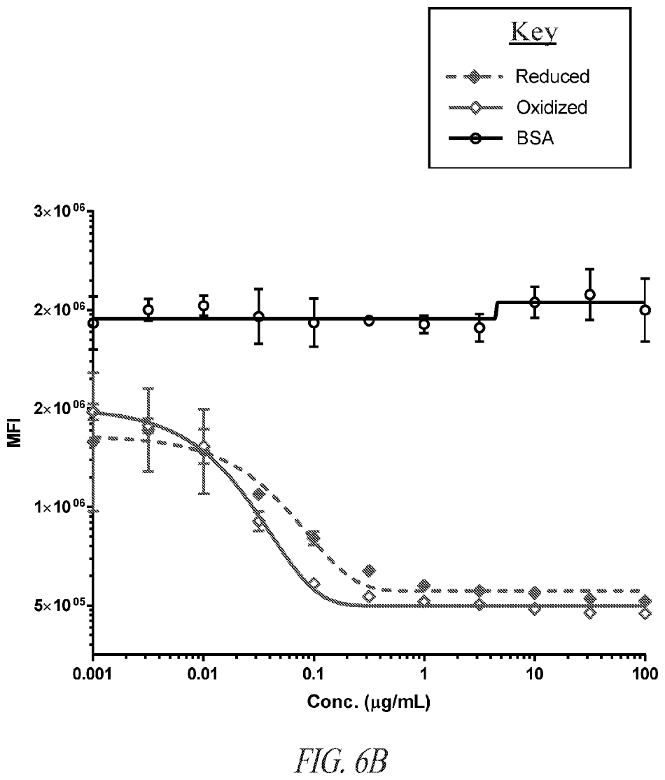

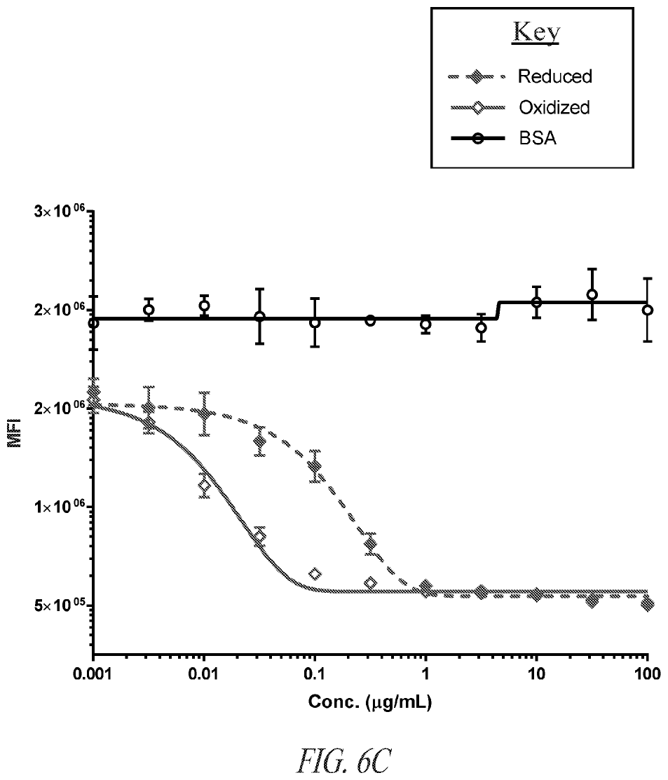

As mentioned above, SEC-MALS analysis suggested that DTT reduction of JO4 has almost no impact on JO4 multimerization and only minimal impact on GB3 multimerization. By contrast, DTT reduction has significant impact on GB7 multimerization and results in significant transition from aggregates/multimers to predominately trimeric forms. To confirm this observation, the viral inhibition assay was performed on reduced and non-reduced (oxidized) proteins (FIG. 6). As shown in FIG. 6a, oxidized and reduced JO4 inhibition curves were nearly indistinguishable from one another and resulted in nearly identical IC50 values. For GB3, reduction resulted in an approximately two-fold reduction in viral inhibition likely due to loss of the trimer-dimer band seen in FIG. 4 (GB3 panel). As expected with GB7, reduction with DTT resulted in a 5-fold decrease in viral inhibition (FIG. 6B) corresponding to the loss of the trimer-dimer peak in GB7 panel of FIG. 4.

A summary of key characteristics of JO4-derivatives GB3 and JOC-x is provided in Table 4.

TABLE 4
Summary of key characteristics of JO4-derivatives

| Characteristic | JO-4 | GB3 | JOC-x |
|---|---|---|---|
| pI | 8.65 | 8.73 | 6.70 |
| Production level (mg/L) | 1-5 | 1-3 | 10-15 |
| Ave. endotoxin (EU/mg) | 691,300K | 80,500 | 2,500 |
| % aggregated by SEC | 90-94 | 43-55 | 4-12 |
| Affinity to DSG2 (nM)² | 0.58 | 0.11 | 1.2 |
| VIA IC₅₀ (μg/mL) | 0.065 | 0.028 | 0.029 |

²Reported values are based on immobilization of DSG2 proteins

### Example 6

### JO-x and Conjugates Exhibit In Vivo Antitumor Activity

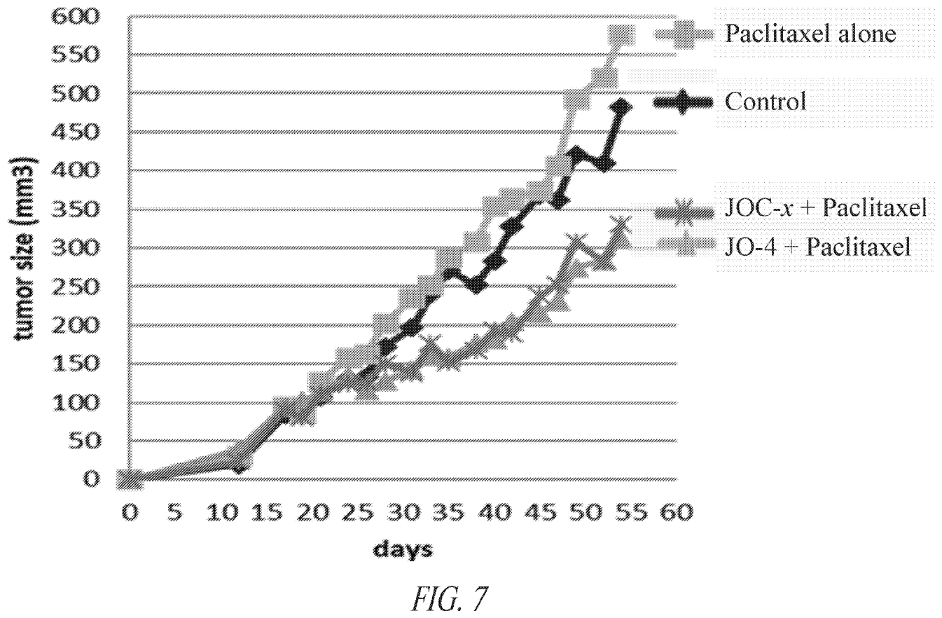

In this Example, JOC-x is shown to have in vivo antitumoral activity. The construct was tested as a mixture with paclitaxel in comparison to JO-4 using a mouse model of cancer (A549 lung cancer). As seen in FIG. 7, although in this experiment the tumor grew quite rapidly, the combination of JOC-x with paclitaxel was equally effective in slowing the growth of the cancer relative to the JO-4 paclitaxel combination. This suggests that the JOC-x structural changes preserved the ability to enhance cancer therapy in vivo.

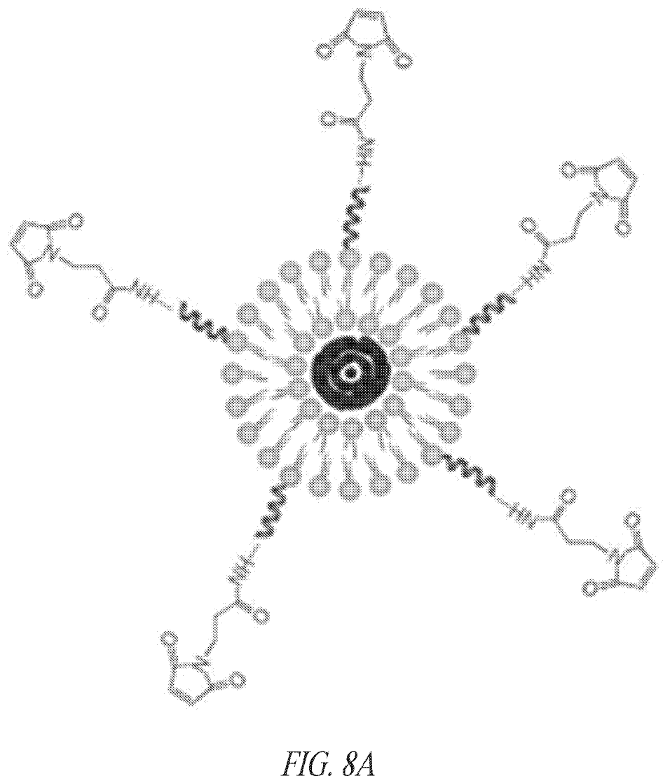

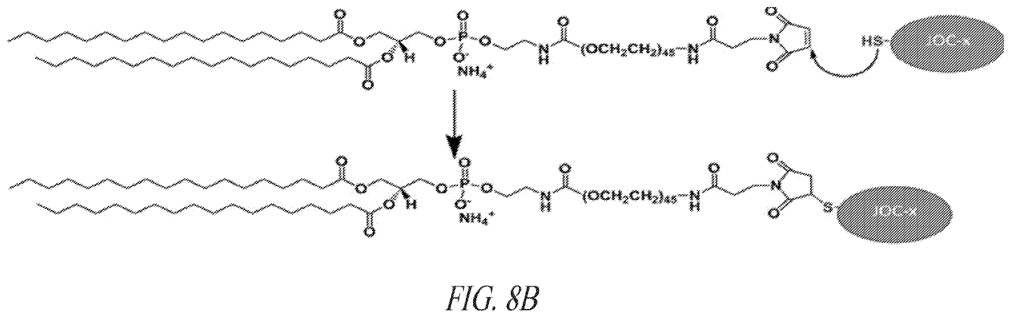

Conjugation of JOC-x with a variety of compounds can improve anti-tumor activity. In an exemplary construct, JOC-x is conjugated with sulfhydryl surface-reactive pegylated doxorubicin liposomes (Doxosome, http://doxosome.com/product-tag/maleimide/), resulting in the structure shown in FIG. 8a. The sulfhydryl reactive forms of drug-loaded liposomes are similar to Doxil®. Additionally, embedded in the liposome is a lipid containing a maleimide group for conjugation to sulfhydryl modified antibodies. Direct conjugation of the free sulfhydryl on JOC-x to PEGylated liposomal doxorubicin by maleimide chemistry is illustrated shown in FIG. 8b. When in contact with JOC-x in the pH range of 6.5-7.5, the maleimide groups on the liposome react with the free sulfhydryl on JOC-x and form a covalent bond. Following full conjugation, each liposome will have multiple JOC proteins attached to facilitate maximum tumor binding and drug delivery. The resulting construct results in a targeted form of Doxil® that will open tumor junctions and deliver the drug preferentially to a solid tumor. Particle size of the liposomes is monitored by dynamic light scattering and mass spectrometry to ensure they remain intact. The resulting reaction mixture may be resolved by SDS-PAGE and mass spectrometry to confirm successful conjugation of JOC-Doxil®. The reaction can be followed spectrophotometrically by the decrease in absorbance at 300 nm as the double bond reacts.

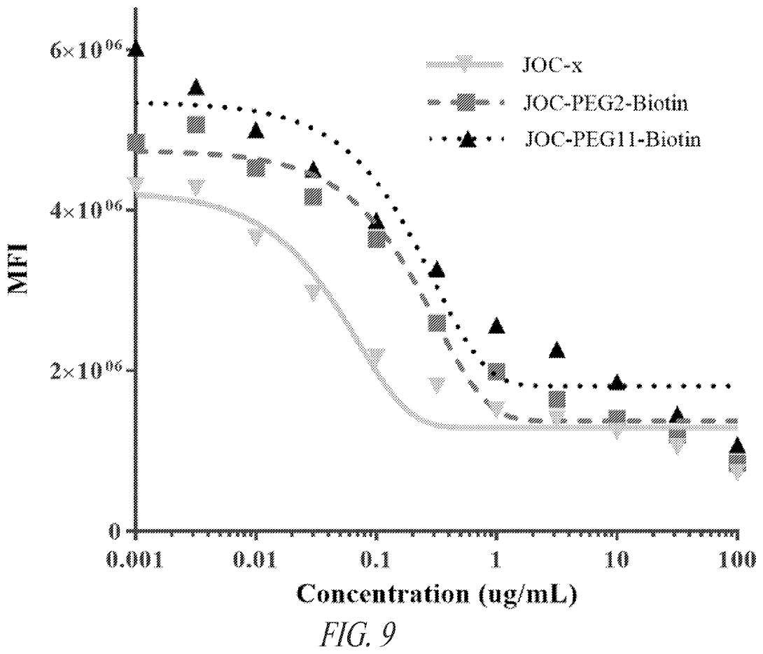

JOC-x can also be conjugated with biotin moieties separated by polyethylene glycol linkers of varying lengths, e.g., PEG2 or PEG11. PEG2-biotin and JOC-PEG11-biotin conjugates are valuable reagents for pre-targeted radioimmunotherapy (PRIT), direct tumor imaging, and immune therapy. PRIT involves injection of a biotinylated tumor-targeting molecule, allowing the molecule to bind its target, and then forming a complex at the site of the tumor following a second injection of streptavidin conjugated to either an imaging agent or radionuclide. In this experiment JOC-x was biotinylated at the terminal cysteinyl residue using the EZ Link Maleimide-PEG2 Biotin or PEG11 Biotin kits (Thermo Scientific, Rockford, Ill.). Using PEG2 or PEG11 spacers assessed whether biotinylation would impair ability of the monomeric protein to form bioactive multimers, and if so, whether increasing the linker length would reduce steric hindrance and increase multimerization and activity. Following biotinylation, the JOC-x and JOC-PEG2/PEG11 conjugates were tested for viral inhibition. As shown in FIG. 9, JOC-PEG2-biotin and JOC-PEG11-biotin both inhibited virus with only slightly impaired IC50 values of 0.125 and 0.203 μg/mL, respectively, compared to 0.032 μg/mL for unconjugated JOC-x. Compared to the IC50 for JOC-x, this represented a 3.9- and 6.3-fold reduction in viral inhibition. This slight impairment of inhibition is not likely to have a significant impact in vivo when used to target streptavidin-conjugated therapies to tumor cells. In addition, conjugation of either PEG2- or PEG11-biotin did not impair the ability of JOC-x to form multimers as evidenced by the formation of both trimers and trimer-dimers in unreduced samples resolved by SDS-PAGE (data not shown).

To produce a conjugate that is directly labeled, less bulky, and more flexible for use in in vivo imaging or radiation of tumors, JOC-x was conjugated to a metal chelation molecule 1,4,7,10-tetraazacyclododecane-1,4,7,10-tetraacetic acid (DOTA). JOC-DOTA conjugates were populated with Europium ions (Eu). DOTA/Eu labeled proteins have been used extensively for in vivo imaging and also have the advantage of being able to sequester radionuclides, such as yttrium-90, used to irradiate targets. In this way, targeted nature of JOC-x to tumors will result in more precisely targeted irradiation of cancer cells while minimizing exposure to healthy tissues.

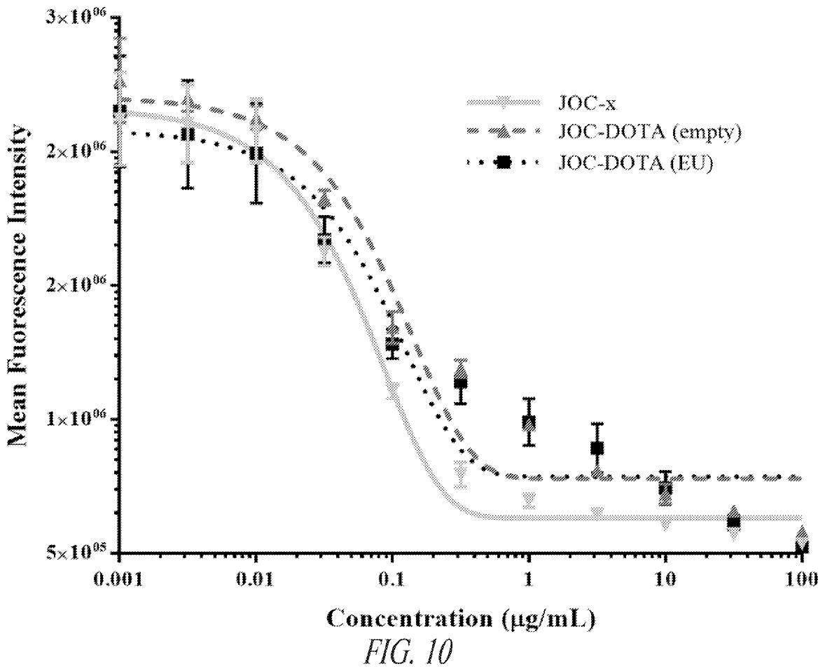

The ability of the conjugates to bind to DSG2 and inhibit viral entry in the VIA assay was assessed. Europium is a rare earth metal and was chosen for two main reasons: 1) it is often used in in vivo imaging due to its phosphorescent properties; and 2) it is a non-radioactive surrogate for radionuclides that may later be used for direct irradiation of tumors, e.g., by populating the DOTA with a radionuclide such as yttrium-90. Because of the small size of DOTA (526 Da), conjugation was confirmed by mass spectrometry (data not shown). The JOC-DOTA conjugate was then complexed with Europium ions to form JOC-DOTA (Eu); successful labeling was demonstrated by reduced and non-reduced SDS-PAGE followed by phosphorescent imaging of the EU ions (data not shown). In this assay, JOC-x, JOC-DOTA (empty), and JOC-DOTA (Eu) were all able to form trimers and trimer-dimers in the unreduced analysis. FIG. 10 shows the results of the viral inhibition assay (VIA) comparing JOC-x, JOC-DOTA (empty), and JOC-DOTA (Eu) for their ability to bind to DSG2 and inhibit viral entry. In VIA, DOTA conjugates maintained their ability to bind DSG2 with IC₅₀ values of 0.055, 0.131, and 0.254 μg/mL for JOC-x, JOC-DOTA, and JOC-DOTA (EU), respectively.

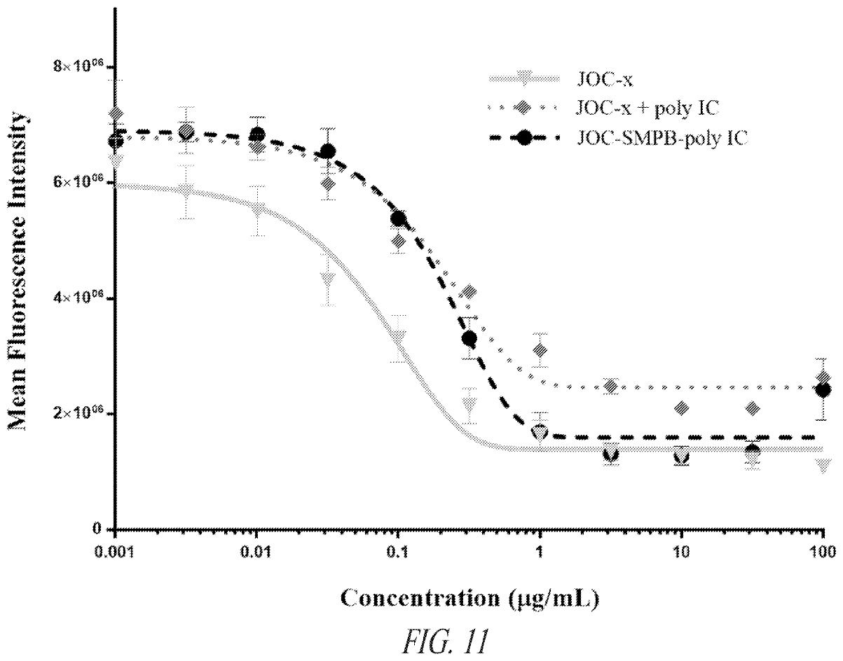

JOC-x was also conjugated to poly(I:C), a potent TLR3 and MDA5 immune stimulator. FIG. 11 shows viral inhibition mediated by the JOC-poly(I:C) (black dashed line, black circles) conjugate compared to either JOC-x alone (grey solid line, grey triangles) or an unconjugated mixture of JOC-x+poly(I:C) (dark grey dotted line, dark grey diamonds). The unconjugated mixture of JOC-x and poly(I:C) measures direct binding of poly(I:C) to a TLR3 positive cell line, HeLa. In HeLa cells TLR3 is primarily found in endosomal compartments and MDA5 intracellular. In the presence of poly(I:C), unconjugated JOC-x exhibited a ˜3.9-fold loss of viral inhibition suggesting binding competition. JOC-poly(I:C) conjugates maintained their ability to bind DSG2 and inhibit viral entry. The slight shift of the inhibition curve to the right is reflective of an approximately 6.3-fold higher IC50 of the conjugate relative to the unconjugated protein (0.032 vs 0.203 μg/mL, respectively). The higher IC50 may be due to steric hindrances imposed by the relatively bulky poly(I:C). Due to the targeted nature of JO, a conjugated JO-poly(I:C) may be a more potent but less toxic form of poly(I:C) for cancer therapy.

Additional characterization of the JOC-poly(I:C) conjugates assessed whether the JOC-poly(I:C) conjugate could trigger innate signaling and activation through viral RNA sensors (TLR3 and MDA5). Poly(I:C) was conjugated to JOC-x using the heterobifunctional crosslinker SMPB [succinimidyl 4-(p-maleimidophenyl) butyrate] linking the free cysteine on JOC-x to the amino terminus of poly(I:C). Signaling and activation were established in vitro by measuring upregulation of production of NF-kB-phosphatase in an HEK-Blue human TLR3 reporter cell line (Invivogen, San Diego, Calif.).

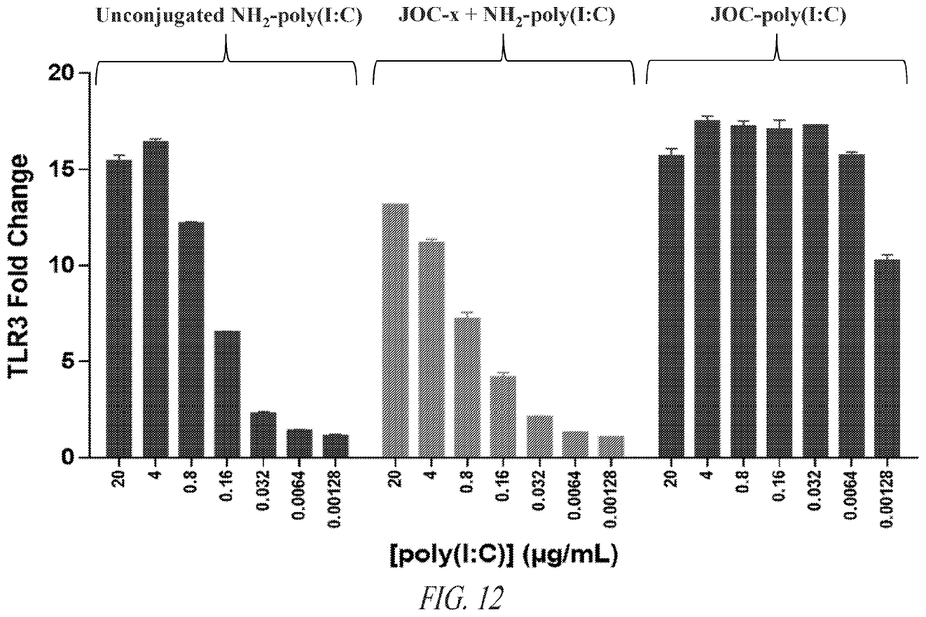

As shown in FIG. 12, JOC-poly(I:C) (far-right bars) was an extremely potent activator of NF-kB-phosphatase. Nearly every concentration of JOC-poly(I:C) induced >17-fold upregulation of TLR3 activation, and at its lowest concentration tested (0.00128 μg/mL), it still achieved >10-fold activation. When an equimolar mixture of the two non-conjugated components were tested (middle light bars), only a 13-fold activation was observed at 20 μg/mL and this showed a rapid dose-dependent decrease <2-fold at 0.032 μg/mL. Poly(I:C) alone (far-left bars) exhibited slightly more activation than the unconjugated mixture with >17-fold activation at 4 μg/mL but also fell quickly to baseline in a dose-dependent manner. At the two lowest tested concentrations, 0.0064 and 0.00128 μg/mL, both poly(I:C) alone and the unconjugated mixture showed no upregulation compared to the JOC-poly(I:C) conjugate which achieved >10-fold activation. These results evidence that the JOC-poly(I:C) conjugate targets TLR3 and MDA5 receptors with significant potency and far better than stimulating TLR3 signaling by poly(I:C) alone. In vivo targeting to DSG2 tumors will allow maximum binding to TLR3 on these cells allowing administration of lower doses (dose-sparing) with less non-specific activation of TLR3 or MDA5 on healthy cells.

All references disclosed herein, including patent references and non-patent references, are hereby incorporated by reference in their entirety as if each was incorporated individually.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include”, as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader, and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means ±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

From the foregoing it will be appreciated that, although specific embodiments have been described herein for purposes of illustration, various modifications may be made without deviating from the spirit and scope of the invention. Accordingly, the invention is not limited except as by the appended claims.

We claim:

1. A polypeptide, comprising:
(a) an adenovirus fiber polypeptide shaft domain motif;
(b) a sequence that induces opening of a tumor tight junction;
(c) a multimerization domain; and
(d) a moiety for targeted conjugation.

2. A polypeptide, comprising:
(a) an adenovirus fiber polypeptide shaft domain motif;
(b) a sequence that induces opening of a tumor tight junction; and
(c) a multimerization domain comprising a conjugatable moiety.

3. The polypeptide of claims 1-2, wherein the sequence that induces opening of a tumor tight junction is a sequence that binds desmoglein-2.

4. The polypeptide of claims 1-2, wherein the shaft domain motif is selected from the group consisting of an Ad3 fiber polypeptide shaft domain motif, an Ad7 fiber polypeptide shaft domain motif, an Ad11 fiber polypeptide shaft domain motif, an Ad 14 fiber polypeptide shaft domain motif, an Ad14a fiber polypeptide shaft domain motif, and combinations thereof.

5. The polypeptide of claim 4, comprising one or more shaft domain motifs having an amino acid sequence selected from the group consisting of SEQ ID NOS: 1-5.

6. The polypeptide of claim 3, wherein the desmoglein 2 binding sequence is an adenovirus knob sequence derived from Ad3, Ad7, Ad11, Ad14, or Ad14a; wherein the desmoglein 2 binding sequence is modified to change or remove amino acids that could compete for conjugation with the conjugatable moiety.

7. The polypeptide of claims 1-2, wherein all cysteinyl residues in the polypeptide other than the conjugatable moiety are changed to serinyl residues.

8. The polypeptide claims 1-2, wherein the conjugatable moiety comprises an amino acid residue that is capable of covalent conjugation.

9. The polypeptide of claims 1-2, wherein the multimerization domain comprises a glycine-serine linker sequence.

10. The polypeptide of claim 8, wherein the amino acid capable of covalent conjugation is a cysteinyl residue.

11. The polypeptide of claim 10, wherein the cysteinyl residue used for conjugation also promotes multimerization.

12. The polypeptide of claim 1 comprising a sequence selected from:
(a)
(SEQ ID NO: 6)
RGSHHHHHHGSKNSIALKNNTLWTGPKPEANSIIEYGKQNPDSKLT
LILVKNGGIVNGYVTLMGASDYVNTLFKNKNVSINVELYFDATGHI
LPDSSSLKTDLELKYKQTADFSARGFMPSTTAYPFDLPNAGTHNEN
YIFGQSYYKASDGALFPLEVTVMLNKRLPDSRTSYVMTFLWSLNAG
LAPETTQATLITSPFTFSYIREDDGGGSGGGSGGGSC;
(b)
(SEQ ID NO: 7)
RGSHHHHHHGSKNSIALKNNTLWTGPKPEANSIIEYGKQNPDSKLT
LILVKNGGIVNGYVTLMGASDYVNTLFKNKNVSINVELYFDATGHI
LPDSSSLKTDLELKYKQTADFSARGFMPSTTAYPFDLPNAGTHNEN
FIFGQSYYKASDGALFPLEVTVMLNKRLPDSRTSYVMTFLWSLNAG
LAPETTQATLITSPFTFSYIREDDGGGSGGGSGGGSC;
(c)
(SEQ ID NO: 8)
RGSHHHHHHGSKNSIALKNNTLWTGPKPEANSIIEYGKQNPDSKLT
LILVKNGGIVNGYVTLMGASDYVNTLFKNKNVSINVELYFDATGHI
LPDSSSLKTDLELKYKQTADFSARGFMPSTTAYPFDLPNAGTHNEN
YIFGQSYYKASDGALFPLEVTVMLNKRLPDSRTSYVMTFLWSLSAG
LAPETTQATLITSPFTFSYIREDDGGGSGGGSGGGSC;
(d)
(SEQ ID NO: 9)
RGSHHHHHHGSKNSIALKNNTLWTGPKPEANSIIEYGKQNPDSKLTL
ILVKNGGIVNGYVTLMGASDYVNTLFKNKNVSINVELYFDATGHILP
DSSSLKTDLELKYKQTADFSARGFMPSTTAYPFDLPNAGTHNENFIF
GQSYYKASDGALFPLEVTVMLNKRLPDSRTSYVMTFLWSLSAGLAPE
TTQATLITSPFTFSYIREDDGGGSGGGSGGGSC;
(e)
(SEQ ID NO: 10)
RGSKNSIALKNNTLWTGPKPEANSIIEYGKQNPDSKLTLILVKNGGI
VNGYVTLMGASDYVNTLFKNKNVSINVELYFDATGHILPDSSSLKTD
LELKYKQTADFSARGFMPSTTAYPFDLPNAGTHNENYIFGQSYYKAS
DGALFPLEVTVMLNKRLPDSRTSYVMTFLWSLNAGLAPETTQATLIT
SPFTFSYIREDDGGGSGGGSGGGSC;
(f)
(SEQ ID NO: 11)
RGSKNSIALKNNTLWTGPKPEANSIIEYGKQNPDSKLTLILVKNGGI
VNGYVTLMGASDYVNTLFKNKNVSINVELYFDATGHILPDSSSLKTD
LELKYKQTADFSARGFMPSTTAYPFDLPNAGTHNENFIFGQSYYKAS
DGALFPLEVTVMLNKRLPDSRTSYVMTFLWSLNAGLAPETTQATLIT
SPFTFSYIREDDGGGSGGGSGGGSC;
(g)
(SEQ ID NO: 12)
RGSKNSIALKNNTLWTGPKPEANSIIEYGKQNPDSKLTLILVKNGGI
VNGYVTLMGASDYVNTLFKNKNVSINVELYFDATGHILPDSSSLKTD
LELKYKQTADFSARGFMPSTTAYPFDLPNAGTHNENYIFGQSYYKAS
DGALFPLEVTVMLNKRLPDSRTSYVMTFLWSLSAGLAPETTQATLIT
SPFTFSYIREDDGGGSGGGSGGGSC;
or
(h)
(SEQ ID NO: 13)
RGSKNSIALKNNTLWTGPKPEANSIIEYGKQNPDSKLTLILVKNGGI
VNGYVTLMGASDYVNTLFKNKNVSINVELYFDATGHILPDSSSLKTD
LELKYKQTADFSARGFMPSTTAYPFDLPNAGTHNENFIFGQSYYKAS
DGALFPLEVTVMLNKRLPDSRTSYVMTFLWSLSAGLAPETTQATLIT
SPFTFSYIREDDGGGSGGGSGGGSC.

13. The polypeptide of claim 1 wherein the multimerization domain functions to conjugates polypeptides to form a multimer.

14. The polypeptide of any one of claims 1-13, further comprising one or more compounds conjugated to the polypeptide.

15. The polypeptide of claim 14, wherein the one or more compounds are selected from the group consisting of therapeutics, diagnostics and imaging agents.

16. The polypeptide of claim 15, wherein the one or more compounds comprise at least one therapeutic, wherein the therapeutic is selected from the group consisting of antibodies, immunoconjugates, immune stimulators, CAR T-cells, nanoparticles, chemotherapeutics, radioactive particles, viruses, vaccines, cellular immunotherapy therapeutics, gene therapy constructs, nucleic acid therapeutics and combinations thereof.

17. An isolated nucleic acid encoding the polypeptide of any one of claims 1-12.

18. A recombinant expression vector comprising the isolated nucleic acid of claim 17.

19. A host cell comprising the recombinant expression vector of claim 18.

20. A pharmaceutical composition, comprising a polypeptide according to any of claims 1-16, and a pharmaceutically acceptable carrier.

21. A method for enhancing therapeutic treatment, or diagnosis of a disorder associated with epithelial tissue, and/or imaging epithelial tissues, comprising administering to a subject in need thereof:
a) a therapeutic for treatment of the disorder, a diagnostic, or an imaging agent; and
(b) the pharmaceutical composition of claim 20, in an amount sufficient to enhance efficacy of the therapeutic, diagnostic, and imaging agent.

22. The method of claim 19, wherein the disorder associated with human tissue is selected from the group consisting of solid tumors, irritable bowel syndrome, inflammatory bowel disorder, Crohn's disease, ulcerative colitis, constipation, gatroesophageal reflux disease, Barrett's esophagus, chronic obstructive pulmonary disease, asthma, bronchitis, pulmonary emphysema, cystic fibrosis, interstitial lung disease, pneumonia, primary pulmonary hypertension, pulmonary embolism, pulmonary sarcoidosis, tuberculosis, pancreatitis, pancreatic duct disorders, bile duct obstruction, cholecystitis, choledocholithiasis, brain disorders, psoriasis, dermatitis, glomerulonephritis, hepatitis, diabetes, thyroid disorders, cellulitis, infection, pyelonephritis, multiple sclerosis, transplant rejection and gallstones.

23. The method of claim 22, wherein the disorder associated with epithelial tissue is a solid tumor.

24. The method of claim 23 wherein the solid tumor is selected from the group consisting of breast tumors, lung tumors, colon tumors, rectal tumors, stomach tumors, prostate tumors, ovarian tumors, uterine tumors, skin tumors, endocrine tumors, cervical tumors, kidney tumors, melanomas, pancreatic tumors, liver tumors, brain tumors, head and neck tumors, nasopharyngeal tumors, gastric tumors, squamous cell carcinomas, adenocarcinomas, bladder tumors and esophageal tumors.

25. The method of any one of claims 21-24 wherein one or more compounds comprises at least one therapeutic, wherein the therapeutic is selected from the group consisting of antibodies, immunoconjugates, immune stimulators, viruses, nanoparticles, chemotherapeutics, radioactive particle, vaccines, cellular immunotherapy therapeutics, gene therapy constructs, nucleic acid therapeutics and combinations thereof.

26. The method of any one of claims 21-24, wherein the therapeutic comprises a chemotherapeutic or a monoclonal antibody.

27. The method of any one of claims 21-24, wherein the therapeutic comprises an anti-tumor monoclonal antibody.

28. The method of claim 27, wherein the anti-tumor monoclonal antibody comprises an antibody selected from the group consisting of trastuzumab, cetuximab, pertuzumab, apomab, conatumumab, lexatumumab, bevacizumab, bevacizumab, denosumab, zanolimumab, lintuzumab, edrecolomab, rituximab, ticilimumab, tositumomab, alemtuzumab, epratuzumab, mitumomab, gemtuzumab ozogamicin, oregovomab, pemtumomab daclizumab, panitumumab, catumaxomab, ofatumumab and ibritumomab.

29. The method of any one of claims 21-27, wherein the disorder associated with epithelial tissue comprises a Her-2 positive tumor.

30. A method for improving delivery of a compound to an epithelial tissue, comprising contacting the epithelial tissue with
(a) one or more compounds to be delivered to the epithelial tissue; and
(b) a conjugated composition of any one of claims 1-16, or functional equivalent thereof, or the pharmaceutical composition of claim 20, sufficient to direct delivery of the one or more compounds to the epithelial tissue.

31. The method of claim 30, wherein the one or more compounds comprises a diagnostic or an imaging agent.

32. The method of claim 30 or 31, wherein the epithelial tissue comprises a solid tumor.

33. The method of claim 30, wherein the solid tumor is selected from the group consisting of breast tumors, lung tumors, colon tumors, rectal tumors, stomach tumors, prostate tumors, ovarian tumors, uterine tumors, skin tumors, endocrine tumors, cervical tumors, kidney tumors, melanomas, pancreatic tumors, liver tumors, brain tumors, head and neck tumors, nasopharyngeal tumors, gastric tumors, squamous cell carcinomas, adenocarcinomas, bladder tumors, and esophageal tumors.

34. A method for improving delivery of a substance to a tissue expressing desmoglein 2 (DSG2), comprising contacting the tissue expressing DSG2 with
(a) one or more compound to be delivered to the tissue; and
(b) linked to an amount of the recombinant protein of any one of claims 1-16, or functional equivalent thereof, or the pharmaceutical composition of claim 20, in an amount sufficient to enhance delivery of the one or more compounds to the tissue.
